(19) United States
(12) Patent Application Publication
(10) Pub. No.: US 2026/0235625 A1
(43) Pub. Date: Aug. 13, 2026

(54) METHODS FOR QUANTITATION OF ADIPONECTIN BY MASS SPECTROMETRY

(71) Applicant: QUEST DIAGNOSTICS INVESTMENTS LLC, Seacaucus, NJ (US)

(72) Inventors: Timothy S. COLLIER, Moreland Hills, OH (US); Jack I. KHAN, Lakewood, OH (US)

(73) Assignee: QUEST DIAGNOSTICS INVESTMENTS LLC, Seacaucus, NJ (US)

(21) Appl. No.: 19/469,665

(22) PCT Filed: Mar. 29, 2024
(86) PCT No.: PCT/US2024/022264

§ 371 Date: Sep. 26, 2025

Related U.S. Application Data
(60) Provisional application No. 63/456,271, filed on Mar. 31, 2023.

(51) Int. Cl. G01N 33/68; G01N 33/74

(52) U.S. Cl. CPC G01N 33/6893; G01N 33/74; G01N 2800/042

(57) ABSTRACT

A method for determining the amount of adiponectin in a sample by mass spectrometry includes (a) subjecting adiponectin from a sample to an ionization source under conditions suitable to generate one or more adiponectin ions detectable by mass spectrometry; (b) determining the amount of one or more adiponectin ions by mass spectrometry; and (c) determining the amount of adiponectin in the sample from the amount of one or more adiponectin ions determined in step (b).

### CROSS REFERENCE TO RELATED APPLICATIONS

This application claims the benefit of priority to U.S. Provisional Patent Application No. 63/456,271, filed Mar. 31, 2023, which is hereby incorporated by reference in its entirety.

### BACKGROUND

Adiponectin is a hormone and an adipokine protein, mainly produced by adipose tissue, that have insulin-sensitizing and anti-inflammatory effects. Individuals with low adiponectin levels have a three times greater risk of developing metabolic syndrome and nine times as likely to develop type 2 diabetes. Men with two or more risk factors for metabolic syndrome and high adiponectin levels are half as likely to develop metabolic syndrome as men with low adiponectin levels. Thus, adiponectin has a significant effect on the metabolic processes.

A reliable and accurate method for measuring adiponectin levels is needed.

### SUMMARY

In one aspect, methods are provided for measuring adiponectin levels in a patient by determining the amount of adiponectin in a sample using mass spectrometry.

In some embodiments, a method for determining the amount of adiponectin in a sample by mass spectrometry is provided, the method including: (a) subjecting adiponectin from a sample to an ionization source under conditions suitable to generate one or more adiponectin ions detectable by mass spectrometry; (b) determining the amount of one or more adiponectin ions by mass spectrometry; and (c) determining the amount of adiponectin in the sample from the amount of one or more adiponectin ions determined in step (b).

In some embodiments, the amount of the one or more ions determined is used to determine the amount of adiponectin in the sample. In some embodiments, the amount of adiponectin in the sample is related to the amount of adiponectin in the patient.

In some embodiments, the sample is a serum sample. In some embodiments, the sample is a plasma sample. In some embodiments, the sample is a blood, saliva, or urine sample.

In some embodiments, the ionization includes electrospray ionization (ESI). In some embodiments, the ionization includes atmospheric pressure chemical ionization (APCI). In some embodiments, the ionization is ionization in positive ion mode. In some embodiments, the ionization is ESI in positive ion mode.

In some embodiments, the one or more adiponectin ions comprise a precursor ion that has a mass to charge ratio (m/z) of 421.23±0.5.

In some embodiments, the one or more adiponectin ions comprise a fragment ion that has a mass to charge ratio (m/z) of 314.18±0.5. In some embodiments, the one or more adiponectin fragment ions comprise an ion that has a mass to charge ratio (m/z) of 627.36±0.5. In some embodiments, the one or more adiponectin fragment ions comprise an ion that has a mass to charge ratio (m/z) of 530.30±0.5.

In some embodiments, methods provided herein comprise adding internal standards to the sample. In some embodiments, an internal standard for adiponectin is added to the sample. In some embodiments, the internal standard is recombinant human adiponectin or adiponectin peptides. In some embodiments, the one or more ions of the internal standard comprise a precursor ion that has a mass to charge ratio (m/z) of 416.22±0.5. In some embodiments, the one or more ions of the internal standard comprise a fragment ion that has a mass to charge ratio (m/z) of 319.19±0.5. In some embodiments, the one or more ions of the internal standard comprise a fragment that has a mass to charge ratio (m/z) of 637.37±0.5. In some embodiments, the one or more ions of the internal standard comprise a fragment that has a mass to charge ratio (m/z) of 540.31±0.5.

In some embodiments, methods provided herein comprise digestion with trypsin. In some embodiments, methods provided herein comprise precipitation with formic acid (e.g., precipitates deoxycholate with formic acid).

In some embodiments, the methods provided herein comprise purifying the samples prior to mass spectrometry. In some embodiments, the methods comprise purifying the samples using liquid chromatography. In some embodiments, liquid chromatography comprise high performance liquid chromatography (HPLC) or high turbulence liquid chromatograph (HTLC). In some embodiments, the methods comprise subjecting a sample to solid phase extraction (SPE).

In some embodiments, the limit of quantitation is less than 2.0 μg/mL. In some embodiments, the limit of quantitation is less than 1.0 μg/mL. In some embodiments, the limit of quantitation is less than 0.9 μg/mL.

In some embodiments, the limit of detection is less than 1.0 μg/mL. In some embodiments, the limit of detection is less than 0.8 μg/mL. In some embodiments, the limit of detection is less than 0.75 μg/mL.

In some embodiments, mass spectrometry comprises tandem mass spectrometry. In some embodiments, mass spectrometry is high resolution mass spectrometry. In some embodiments, mass spectrometry is high resolution/high accuracy mass spectrometry.

In some embodiments, a low level (e.g., below the established reference ranges described in Table A) of adiponectin corresponds to increased risk of metabolic syndrome. In some embodiments, a low level (e.g., below the established reference ranges described in Table A) of adiponectin corresponds to increased risk of type 2 diabetes.

In some embodiments, the collision energy is within the range of about 5V to about 25 V. In some embodiments, the collision energy is within the range of about 9V to about 21 V. In some embodiments, the collision energy is about 9 V. In some embodiments, the collision energy is about 21V.

In another aspect, provided herein are methods for diagnosis of glycemic disorders or insulin resistant syndromes in diabetic and pre-diabetic patients. In some embodiments, the methods of quantitation of endogenous adiponectin provided herein are used for diagnosing diabetes. In some embodiments, the methods of quantitation of endogenous adiponectin provided herein are used for distinguishing type 1 diabetes from type 2 diabetes. In some embodiments, the methods of quantitation of endogenous adiponectin provided herein are used for assessing the risk of diabetes in pre-diabetic patients.

In another aspect, provided herein are methods for diagnosis or prognosis of glycemic disorders or insulin resistant syndromes in diabetic and pre-diabetic patients comprising comparing the relative amount of adiponectin. In another aspect, provided herein are methods for diagnosis or prognosis of glycemic disorders or insulin resistant syndromes in diabetic and pre-diabetic patients comprising determining the levels of adiponectin. In some embodiments, the methods comprise determining the amount of adiponectin. In some embodiments, an aberrant or abnormal amount of adiponectin indicates glycemic disorders or insulin resistant syndromes. In some embodiments, a low level (e.g., below the established reference ranges described in Table A) of adiponectin indicates glycemic disorders or insulin resistant syndromes.

The summary of the invention described above is non-limiting and other features and advantages of the invention will be apparent from the following detailed description of the invention, and from the claims.

### BRIEF DESCRIPTION OF THE DRAWINGS

FIGS. 1A-1B show the comparison between the validated ELISA method and LC/MS method yielded a slope of 1.008 with an intercept of −0.167 (FIG. 1A) and difference plot between ELISA and LC/MS (FIG. 1B).

FIGS. 2A-2C show all three levels of QC meet total precision acceptance criteria of TEa/3 at low (FIG. 2A), mid (FIG. 2B), and high (FIG. 2C).

FIG. 3 shows that acceptance criteria was met across the AMR from 1.50 ug/mL to 50.00 g/mL.

FIGS. 4A-4B show carryover and pre-carryover did not exceed TEa/4 for adiponectin, thus passing acceptance criteria at LC3 (FIG. 4A) and at LC4 (FIG. 4B).

FIG. 5 shows that LOB was calculated to be 0.419 μg/mL, LOD of 0.740 ug/mL, and LOQ of 0.831 μg/mL. Each of the low pools had a SD<TEa/3 and passed acceptance criteria.

FIG. 6 shows that the average concentration was within TEa/3 of the average concentration measured at t=0 for all assessed timepoints.

FIG. 7 shows that the average concentration was within TEa/3 of the average concentration measured at t=0 for all assessed timepoints.

FIG. 8 shows that the average concentration was within TEa/3 of the average concentration measured at t=0 for all time points up to 20 days.

FIGS. 9A-9B show the average concentration was within TEa/3 of the average concentration measured at t=0 for all assessed timepoints at-20 to-40C (FIG. 9A) and at-70° C. (FIG. 9B).

FIG. 10 shows that the average concentration was within TEa/3 of the average concentration measured at t=0 for all assessed freeze/thaw cycles.

FIGS. 11A-11B show plate stability (cold stack) with adiponectin comparison (FIG. 11A) and adiponectin difference plot (FIG. 11B).

FIGS. 12A-12B show hemolysis interference (FIG. 12A) and hemolysate (FIG. 12B).

FIGS. 13A-13B show icterus (bilirubin) interference (FIG. 13A) and bilirubin (FIG. 13B).

FIGS. 14A-14B show lipemia (intralipid) interference (FIG. 14A) and intralipid (FIG. 14B).

### DETAILED DESCRIPTION

As used herein, unless otherwise stated, the singular forms “a,” “an,” and “the” include plural reference. Thus, for example, a reference to “a protein” includes a plurality of protein molecules.

As used herein, the terms “purification”, “purifying”, and “enriching” do not refer to removing all materials from the sample other than the analyte(s) of interest. Instead, these terms refer to a procedure that enriches the amount of one or more analytes of interest relative to other components in the sample that may interfere with detection of the analyte of interest. Purification of the sample by various means may allow relative reduction of one or more interfering substances, e.g., one or more substances that may or may not interfere with the detection of selected parent or daughter ions by mass spectrometry. Relative reduction as this term is used does not require that any substance, present with the analyte of interest in the material to be purified, is entirely removed by purification.

As used herein, the term “immunopurification” or “immunopurify” refers to a purification procedure that utilizes antibodies, including polyclonal or monoclonal antibodies, to enrich the one or more analytes of interest. Immunopurification can be performed using any of the immunopurification methods well known in the art. Often the immunopurification procedure utilizes antibodies bound, conjugated or otherwise attached to a solid support, for example a column, well, tube, gel, capsule, particle or the like. Immunopurification as used herein includes without limitation procedures often referred to in the art as immunoprecipitation, as well as procedures often referred to in the art as affinity chromatography or immunoaffinity chromatography.

As used herein, the term “immunoparticle” refers to a capsule, bead, gel particle or the like that has antibodies bound, conjugated or otherwise attached to its surface (either on and/or in the particle). In certain preferred embodiments, immunoparticles are sepharose or agarose beads. In alternative preferred embodiments, immunoparticles comprise glass, plastic or silica beads, or silica gel.

As used herein, the term “anti-adiponectin antibody” refers to any polyclonal or monoclonal antibody that has an affinity for adiponectin. In various embodiments the specificity of adiponectin antibodies to chemical species other than adiponectin may vary; for example in certain preferred embodiments the anti-adiponectin antibodies are specific for adiponectin and thus have little or no affinity for chemical species other than adiponectin.

As used herein, the term “sample” refers to any sample that may contain an analyte of interest. As used herein, the term “body fluid” means any fluid that can be isolated from the body of an individual. For example, “body fluid” may include blood, plasma, serum, bile, saliva, urine, tears, perspiration, and the like. In preferred embodiments, the sample comprises a body fluid sample from human; preferably plasma or serum.

As used herein, the term “solid phase extraction” or “SPE” refers to a process in which a chemical mixture is separated into components as a result of the affinity of components dissolved or suspended in a solution (i.e., mobile phase) for a solid through or around which the solution is passed (i.e., solid phase). In some instances, as the mobile phase passes through or around the solid phase, undesired components of the mobile phase may be retained by the solid phase resulting in a purification of the analyte in the mobile phase. In other instances, the analyte may be retained by the solid phase, allowing undesired components of the mobile phase to pass through or around the solid phase. In these instances, a second mobile phase is then used to elute the retained analyte off of the solid phase for further processing or analysis. SPE, including TFLC, may operate via a unitary or mixed mode mechanism. Mixed mode mechanisms utilize ion exchange and hydrophobic retention in the same column; for example, the solid phase of a mixed-mode SPE column may exhibit strong anion exchange and hydrophobic retention; or may exhibit strong cation exchange and hydrophobic retention.

Generally, the affinity of a SPE column packing material for an analyte may be due to any of a variety of mechanisms, such as one or more chemical interactions or an immunoaffinity interaction. In some embodiments, SPE of adiponectin is conducted without the use of an immunoaffinity column packing material. That is, in some embodiments, adiponectin is purified from a sample by a SPE column that is not an immunoaffinity column.

As used herein, the term “chromatography” refers to a process in which a chemical mixture carried by a liquid or gas is separated into components as a result of differential distribution of the chemical entities as they flow around or over a stationary liquid or solid phase.

As used herein, the term “liquid chromatography” or “LC” means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Examples of “liquid chromatography” include reverse phase liquid chromatography (RPLC), high performance liquid chromatography (HPLC), and turbulent flow liquid chromatography (TFLC) (sometimes known as high turbulence liquid chromatography (HTLC) or high throughput liquid chromatography).

As used herein, the term “high performance liquid chromatography” or “HPLC” (sometimes known as “high pressure liquid chromatography”) refers to liquid chromatography in which the degree of separation is increased by forcing the mobile phase under pressure through a stationary phase, typically a densely packed column.

As used herein, the term “turbulent flow liquid chromatography” or “TFLC” (sometimes known as high turbulence liquid chromatography or high throughput liquid chromatography) refers to a form of chromatography that utilizes turbulent flow of the material being assayed through the column packing as the basis for performing the separation. TFLC has been applied in the preparation of samples containing two unnamed drugs prior to analysis by mass spectrometry. See, e.g., Zimmer et al., J Chromatogr A 854:23-35 (1999); see also, U.S. Pat. Nos. 5,968,367, 5,919,368, 5,795,469, and 5,772,874, which further explain TFLC. Persons of ordinary skill in the art understand “turbulent flow”. When fluid flows slowly and smoothly, the flow is called “laminar flow”. For example, fluid moving through an HPLC column at low flow rates is laminar. In laminar flow the motion of the particles of fluid is orderly with particles moving generally in substantially straight lines. At faster velocities, the inertia of the water overcomes fluid frictional forces and turbulent flow results. Fluid not in contact with the irregular boundary “outruns” that which is slowed by friction or deflected by an uneven surface. When a fluid is flowing turbulently, it flows in eddies and whirls (or vortices), with more “drag” than when the flow is laminar. Many references are available for assisting in determining when fluid flow is laminar or turbulent (e.g., Turbulent Flow Analysis: Measurement and Prediction, P. S. Bernard & J. M. Wallace, John Wiley & Sons, Inc., (2000); An Introduction to Turbulent Flow, Jean Mathieu & Julian Scott, Cambridge University Press (2001)).

As used herein, the term “gas chromatography” or “GC” refers to chromatography in which the sample mixture is vaporized and injected into a stream of carrier gas (as nitrogen or helium) moving through a column containing a stationary phase composed of a liquid or a particulate solid and is separated into its component compounds according to the affinity of the compounds for the stationary phase.

As used herein, the term “large particle column” or “extraction column” refers to a chromatography column containing an average particle diameter greater than about 50 μm. As used in this context, the term “about” means±10%.

As used herein, the term “analytical column” refers to a chromatography column having sufficient chromatographic plates to effect a separation of materials in a sample that elute from the column sufficient to allow a determination of the presence or amount of an analyte. Such columns are often distinguished from “extraction columns”, which have the general purpose of separating or extracting retained material from non-retained materials to obtain a purified sample for further analysis. As used in this context, the term “about” means±10%. In a preferred embodiment the analytical column contains particles of about 5 μm in diameter.

As used herein, the terms “on-line” and “inline”, for example as used in “on-line automated fashion” or “on-line extraction”, refers to a procedure performed without the need for operator intervention. In contrast, the term “off-line” as used herein refers to a procedure requiring manual intervention of an operator. Thus, if samples are subjected to precipitation and the supernatants are then manually loaded into an autosampler, the precipitation and loading steps are off-line from the subsequent steps. In various embodiments of the methods, one or more steps may be performed in an on-line automated fashion.

As used herein, the term “mass spectrometry” or “MS” refers to an analytical technique to identify compounds by their mass. MS refers to methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer, a mass analyzer, and an ion detector. In general, one or more molecules of interest are ionized, and the ions are subsequently introduced into a mass spectrometric instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces;” 6,107,623, entitled “Methods and Apparatus for Tandem Mass Spectrometry;” 6,268,144, entitled “DNA Diagnostics Based On Mass Spectrometry;” 6,124, 137, entitled “Surface-Enhanced Photolabile Attachment And Release For Desorption And Detection Of Analytes;” Wright et al., Prostate Cancer and Prostatic Diseases 1999, 2:264-76; and Merchant and Weinberger, Electrophoresis 2000, 21:1164-67.

As used herein, “high resolution/high accuracy mass spectrometry” refers to mass spectrometry conducted with a mass analyzer capable of measuring the mass to charge ratio of a charged species with sufficient precision and accuracy to confirm a unique chemical ion. Confirmation of a unique chemical ion is possible for an ion when individual isotopic peaks from that ion are readily discernable. The particular resolving power and mass accuracy necessary to confirm a unique chemical ion varies with the mass and charge state of the ion.

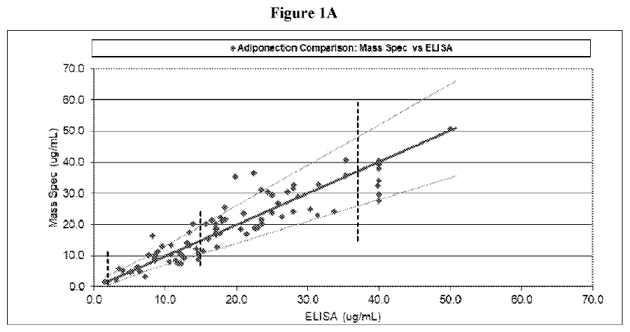

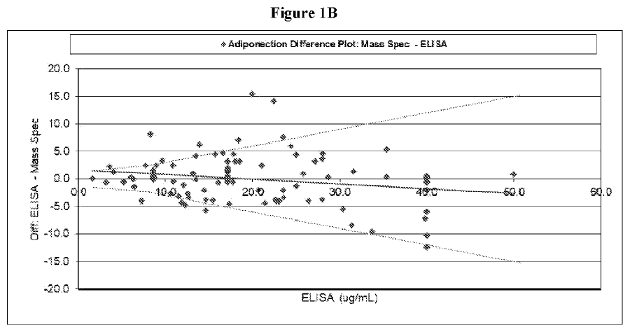

As used herein, the term “resolving power” or “resolving power (FWHM)” (also known in the art as “m/Δm_{50%}”) refers to an observed mass to charge ratio divided by the width of the mass peak at 50% maximum height (Full Width Half Maximum, “FWHM”). The effect of differences in resolving power is illustrated in FIGS. 1A-C, which show theoretical mass spectra of an ion with a m/z of about 1093. FIG. 1A shows a theoretical mass spectrum from a mass analyzer with resolving power of about 3000 (a typical operating condition for a conventional quadrupole mass analyzer). As seen in FIG. 1A, no individual isotopic peaks are discernable. By comparison, FIG. 1B shows a theoretical mass spectrum from a mass analyzer with resolving power of about 10,000, with clearly discernable individual isotopic peaks. FIG. 1C shows a theoretical mass spectrum from a mass analyzer with resolving power of about 12,000. At this highest resolving power, the individual isotopic peaks contain less than 1% contribution from baseline.

As used herein a “unique chemical ion” with respect to mass spectrometry refers a single ion with a single atomic makeup. The single ion may be singly or multiply charged.

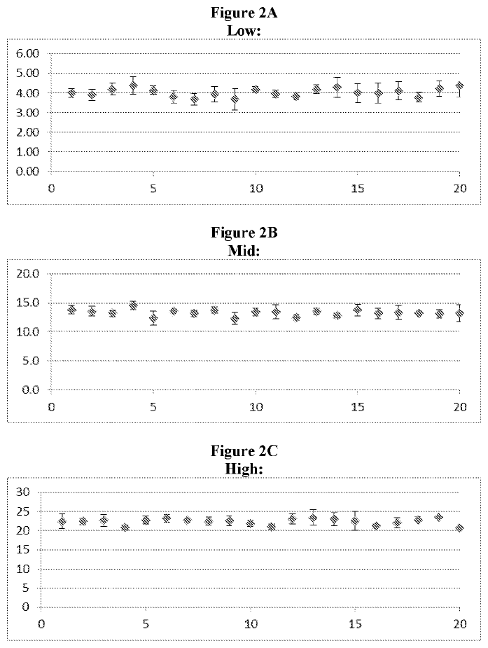

As used herein, the term “accuracy” (or “mass accuracy”) with respect to mass spectrometry refers to potential deviation of the instrument response from the true m/z of the ion investigated. Accuracy is typically expressed in parts per million (ppm). The effect of differences in mass accuracy is illustrated in FIGS. 2A-D, which show the boundaries of potential differences between a detected m/z and the actual m/z for a theoretical peak at m/z of 1093.52094. FIG. 2A shows the potential range of detected m/z at an accuracy of 120 ppm. By contrast, FIG. 2B shows the potential range of detected m/z at an accuracy of 50 ppm. FIGS. 2C and 2D show the even narrower potential ranges of detected m/z at accuracies of 20 ppm and 10 ppm.

High resolution/high accuracy mass spectrometry methods of the present invention may be conducted on instruments capable of performing mass analysis with FWHM of greater than 10,000, 15,000, 20,000, 25,000, 50,000, 100,000, or even more. Likewise, methods of the present invention may be conducted on instruments capable of performing mass analysis with accuracy of less than 50 ppm, 20 ppm, 15 ppm, 10 ppm, 5 ppm, 3 ppm, or even less. Instruments capable of these performance characteristics may incorporate certain orbitrap mass analyzers, time-of-flight (“TOF”) mass analyzers, or Fourier-transform ion cyclotron resonance mass analyzers. In preferred embodiments, the methods are carried out with an instrument which includes an orbitrap mass analyzer or a TOF mass analyzer.

The term “orbitrap” describes an ion trap consisting of an outer barrel-like electrode and a coaxial inner electrode. Ions are injected tangentially into the electric field between the electrodes and trapped because electrostatic interactions between the ions and electrodes are balanced by centrifugal forces as the ions orbit the coaxial inner electrode. As an ion orbits the coaxial inner electrode, the orbital path of a trapped ion oscillates along the axis of the central electrode at a harmonic frequency relative to the mass to charge ratio of the ion. Detection of the orbital oscillation frequency allows the orbitrap to be used as a mass analyzer with high accuracy (as low as 1-2 ppm) and high resolving power (FWHM) (up to about 200,000). A mass analyzer based on an orbitrap is described in detail in U.S. Pat. No. 6,995,364, incorporated by reference herein in its entirety. Use of orbitrap analyzers has been reported for qualitative and quantitative analyses of various analytes. See, e.g., U.S. Patent Application Pub. No. 2008/0118932 (filed Nov. 9, 2007); Bredehoft, et al., Rapid Commun. Mass Spectrom., 2008, 22:477-485; Le Breton, et al., Rapid Commun. Mass Spectrom., 2008, 22:3130-36; Thevis, et al., Mass Spectrom. Reviews, 2008, 27:35-50; Thomas, et al., J. Mass Spectrom., 2008, 43:908-15; Schenk, et al., BMC Medical Genomics, 2008, 1:41; and Olsen, et al., Nature Methods, 2007, 4:709-12.

As used herein, the term “operating in negative ion mode” refers to those mass spectrometry methods where negative ions are generated and detected. The term “operating in positive ion mode” as used herein, refers to those mass spectrometry methods where positive ions are generated and detected. In preferred embodiments, mass spectrometry is conducted in positive ion mode.

As used herein, the term “ionization” or “ionizing” refers to the process of generating an analyte ion having a net electrical charge equal to one or more electron units. Negative ions are those having a net negative charge of one or more electron units, while positive ions are those having a net positive charge of one or more electron units.

As used herein, the term “electron ionization” or “EI” refers to methods in which an analyte of interest in a gaseous or vapor phase interacts with a flow of electrons. Impact of the electrons with the analyte produces analyte ions, which may then be subjected to a mass spectrometry technique.

As used herein, the term “chemical ionization” or “CI” refers to methods in which a reagent gas (e.g. ammonia) is subjected to electron impact, and analyte ions are formed by the interaction of reagent gas ions and analyte molecules.

As used herein, the term “fast atom bombardment” or “FAB” refers to methods in which a beam of high energy atoms (often Xe or Ar) impacts a non-volatile sample, desorbing and ionizing molecules contained in the sample. Test samples are dissolved in a viscous liquid matrix such as glycerol, thioglycerol, m-nitrobenzyl alcohol, 18-crown-6 crown ether, 2-nitrophenyloctyl ether, sulfolane, diethanolamine, and triethanolamine. The choice of an appropriate matrix for a compound or sample is an empirical process.

As used herein, the term “matrix-assisted laser desorption ionization” or “MALDI” refers to methods in which a non-volatile sample is exposed to laser irradiation, which desorbs and ionizes analytes in the sample by various ionization pathways, including photo-ionization, protonation, deprotonation, and cluster decay. For MALDI, the sample is mixed with an energy-absorbing matrix, which facilitates desorption of analyte molecules.

As used herein, the term “surface enhanced laser desorption ionization” or “SELDI” refers to another method in which a non-volatile sample is exposed to laser irradiation, which desorbs and ionizes analytes in the sample by various ionization pathways, including photo-ionization, protonation, deprotonation, and cluster decay. For SELDI, the sample is typically bound to a surface that preferentially retains one or more analytes of interest. As in MALDI, this process may also employ an energy-absorbing material to facilitate ionization.

As used herein, the term “electrospray ionization” or “ESI,” refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber. As the droplets get smaller the electrical surface charge density increases until such time that the natural repulsion between like charges causes ions as well as neutral molecules to be released.

As used herein, the term “atmospheric pressure chemical ionization” or “APCI,” refers to mass spectrometry methods that are similar to ESI; however, APCI produces ions by ion-molecule reactions that occur within a plasma at atmospheric pressure. The plasma is maintained by an electric discharge between the spray capillary and a counter electrode. Then ions are typically extracted into the mass analyzer by use of a set of differentially pumped skimmer stages. A counterflow of dry and preheated N₂ gas may be used to improve removal of solvent. The gas-phase ionization in APCI can be more effective than ESI for analyzing less-polar species.

The term “atmospheric pressure photoionization” or “APPI” as used herein refers to the form of mass spectrometry where the mechanism for the ionization of molecule M is photon absorption and electron ejection to form the molecular ion M+. Because the photon energy typically is just above the ionization potential, the molecular ion is less susceptible to dissociation. In many cases it may be possible to analyze samples without the need for chromatography, thus saving significant time and expense. In the presence of water vapor or protic solvents, the molecular ion can extract H to form MH+. This tends to occur if M has a high proton affinity. This does not affect quantitation accuracy because the sum of M+ and MH+ is constant. Drug compounds in protic solvents are usually observed as MH+, whereas nonpolar compounds such as naphthalene or testosterone usually form M+. See, e.g., Robb et al., Anal. Chem. 2000, 72 (15): 3653-3659.

As used herein, the term “inductively coupled plasma” or “ICP” refers to methods in which a sample interacts with a partially ionized gas at a sufficiently high temperature such that most elements are atomized and ionized.

As used herein, the term “field desorption” refers to methods in which a non-volatile test sample is placed on an ionization surface, and an intense electric field is used to generate analyte ions.

As used herein, the term “desorption” refers to the removal of an analyte from a surface and/or the entry of an analyte into a gaseous phase. Laser desorption thermal desorption is a technique wherein a sample containing the analyte is thermally desorbed into the gas phase by a laser pulse. The laser hits the back of a specially made 96-well plate with a metal base. The laser pulse heats the base and the heat causes the sample to transfer into the gas phase. The gas phase sample is then drawn into the mass spectrometer.

As used herein, the term “selective ion monitoring” is a detection mode for a mass spectrometric instrument in which only ions within a relatively narrow mass range, typically about one mass unit, are detected.

As used herein, “multiple reaction mode,” sometimes known as “selected reaction monitoring,” is a detection mode for a mass spectrometric instrument in which a precursor ion and one or more fragment ions are selectively detected.

As used herein, the term “lower limit of quantification”, “lower limit of quantitation” or “LLOQ” refers to the point where measurements become quantitatively meaningful. The analyte response at this LOQ is identifiable, discrete and reproducible with a relative standard deviation (RSD %) of less than 20% and an accuracy of 85% to 115%.

As used herein, the term “limit of detection” or “LOD” is the point at which the measured value is larger than the uncertainty associated with it. The LOD is the point at which a value is beyond the uncertainty associated with its measurement and is defined as three times the RSD of the mean at the zero concentration.

As used herein, an “amount” of an analyte in a body fluid sample refers generally to an absolute value reflecting the mass of the analyte detectable in volume of sample. However, an amount also contemplates a relative amount in comparison to another analyte amount. For example, an amount of an analyte in a sample can be an amount which is greater than a control or normal level of the analyte normally present in the sample.

The term “about” as used herein in reference to quantitative measurements not including the measurement of the mass of an ion, refers to the indicated value plus or minus 10%. Mass spectrometry instruments can vary slightly in determining the mass of a given analyte. The term “about” in the context of the mass of an ion or the mass/charge ratio of an ion refers to +/−0.50 atomic mass unit.

In one aspect, provided herein are methods for measuring adiponectin levels in a patient by determining the amount of adiponectin in a sample using mass spectrometry.

The methods may be for determining the amount of adiponectin in a sample by mass spectrometry, the method comprising: (a) subjecting adiponectin from a sample to an ionization source under conditions suitable to generate one or more adiponectin ions detectable by mass spectrometry; (b) determining the amount of one or more adiponectin ions by mass spectrometry; and (c) determining the amount of the amount of adiponectin in the sample from the amount of the amount of one or more adiponectin ions determined in step (b)

The amount of the one or more ions determined may then be used to determine the amount of adiponectin in the sample. In some embodiments, the amount of adiponectin in the sample is related to the amount of adiponectin in the patient.

The sample may be a serum sample. In some embodiments, the sample is a plasma sample. In some embodiments, the sample is a blood, saliva, or urine sample.

The ionization may include electrospray ionization (ESI). In some embodiments, the ionization includes atmospheric pressure chemical ionization (APCI). In some embodiments, the ionization is in positive ion mode. In some embodiments, the ionization is ESI in positive ion mode.

The one or more adiponectin ions may include a precursor ion that has a mass to charge ratio (m/z) of 421.23±0.5. The one or more adiponectin ions may also include a fragment ion that has a mass to charge ratio (m/z) of 314.18±0.5. In some embodiments, the one or more adiponectin fragment ions may include an ion that has a mass to charge ratio (m/z) of 627.36±0.5. In some embodiments, the one or more adiponectin fragment ions may include an ion that has a mass to charge ratio (m/z) of 530.30±0.5.

The methods provided herein may include adding internal standards to the sample. The internal standard for adiponectin may be added to the sample. In some embodiments, the internal standard is recombinant human adiponectin or adiponectin peptides. The methods may also include generating one or more ions of the internal standard that are detectable by mass spectrometry. The one or more ions of the internal standard may include a precursor ion that has a mass to charge ratio (m/z) of 416.22±0.5. In some embodiments, the one or more ions of the internal standard may include a fragment ion that has a mass to charge ratio (m/z) of 319.19±0.5. In some embodiments, the one or more ions of the internal standard may include a fragment that has a mass to charge ratio (m/z) of 637.37±0.5. In some embodiments, the one or more ions of the internal standard may include a fragment that has a mass to charge ratio (m/z) of 540.31±0.5.

The methods provided herein may include digesting adiponectin in the sample. The digestion may be accomplished using trypsin in some embodiments. Further, the methods may include subjecting the sample (e.g., a solution) with adiponectin (and/or a digestion product of adiponectin) to precipitation (e.g., precipitates deoxycholate with formic acid). In some embodiments, the sample (e.g., the solution) comprising adiponectin (and/or a digestion product of adiponectin) may be purified by precipitation (e.g., precipitation with formic acid).

The methods provided herein may also purifying the samples prior to mass spectrometry. Such purification may be done using liquid chromatography, which may, in some embodiments, be high performance liquid chromatography (HPLC) or high turbulence liquid chromatography (HTLC). The methods may also include subjecting a sample to solid phase extraction (SPE).

Various mass spectroscopy techniques may be used such as tandem mass spectrometry, high resolution mass spectrometry, or high resolution/high accuracy mass spectrometry.

The methods may provide for a limit of quantitation of less than 2.0 μg/mL. This may include where the limit of quantitation of the method is less than 1.0 μg/mL, less than 0.9 μg/mL, less than 0.8 μg/mL, or less than 0.75 μg/mL according to various embodiments.

Low levels of adiponectin may corresponds to an increased risk of metabolic syndrome. For example, a low level of adiponectin may correspond to an increased risk of type 2 diabetes. As used herein, term “low level” (e.g., a “low level” of adiponectin) generally refers to an amount that is below the established reference range of an analyte as determined from a cohort of apparently healthy individuals. For example, without being bound by theory, the reference ranges of adiponectin are believed to be those as shown in Table A, which are sorted by patient sex and body mass index (BMI).

TABLE A
Reference ranges of adiponectin in healthy individuals

| | | |
|---|---|---|
| BMI | Male | Female |

| | | | |
|---|---|---|---|
| <25 | kg/m² | 4-26 μg/mL | 5-37 μg/mL |
| 25-30 | kg/m² | 4-20 μg/mL | 5-28 μg/mL |
| >30 | kg/m² | 2-20 μg/mL | 4-22 μg/mL |

In some embodiments, the collision energy used in a method is from about 5V to about 25 V. This may include where the collision energy is from about 9V to about 21 V, is about 9 V, or is about 21V.

In another aspect, methods for diagnosis of glycemic disorders or insulin resistant syndromes in diabetic and pre-diabetic patients are provided. Such methods of quantitation of endogenous adiponectin may be used for diagnosing diabetes, or for distinguishing type 1 diabetes from type 2 diabetes. In some embodiments, the methods of quantitation of endogenous adiponectin are used for assessing the risk of diabetes in pre-diabetic patients.

In another aspect, methods are provided for the diagnosis of, or determining the prognosis for, glycemic disorders or insulin resistant syndromes in diabetic and pre-diabetic patients. Such methods may include comparing the relative amount of adiponectin (e.g., by comparing the amount of adiponectin in a patient to the corresponding reference described in Table A).

In another aspect, methods are provided for the diagnosis of, or determining the prognosis for, glycemic disorders or insulin resistant syndromes in diabetic and pre-diabetic patients. Such methods include determining levels of adiponectin in a sample from a subject. The methods may include determining the amount of adiponectin. In some embodiments, an aberrant or abnormal amount of adiponectin indicates glycemic disorders or insulin resistant syndromes. In other embodiments, a low level (e.g., below the established references described in Table A) of adiponectin indicates glycemic disorders or insulin resistant syndromes.

In a further aspect, kits are provided for an adiponectin quantitation assay. A kit for an adiponectin quantitation assay may include a kit comprising the compositions provided herein. For example, a kit may include packaging material and measured amounts of an isotopically labeled internal standard, in amounts sufficient for at least one assay. Typically, the kits will also include instructions recorded in a tangible form (e.g., contained on paper or an electronic medium) for using the packaged reagents for use in an adiponectin quantitation assay.

Calibration and QC (quality control) pools for use in embodiments of the present invention are preferably prepared using a matrix similar to the intended sample matrix, provided that adiponectin is essentially absent.

### Sample Preparation for Mass Spectrometric Analysis

In preparation for mass spectrometric analysis, adiponectin may be enriched relative to one or more other components in the sample by various methods known in the art, including for example, immunocapture, liquid chromatography, filtration, centrifugation, thin layer chromatography (TLC), electrophoresis including capillary electrophoresis, affinity separations including immunoaffinity separations, extraction methods including ethyl acetate or methanol extraction, and the use of chaotropic agents or any combination of the above or the like.

One method of sample purification that may be used prior to mass spectrometry is applying a sample to a solid-phase extraction (SPE) column under conditions where the analyte of interest is reversibly retained by the column packing material, while one or more other materials are not retained. In this technique, a first mobile phase condition can be employed where the analyte of interest is retained by the column, and a second mobile phase condition can subsequently be employed to remove retained material from the column, once the non-retained materials are washed through.

In some embodiments, adiponectin in a sample may be reversibly retained on a SPE column with a packing material comprising an alkyl bonded surface. For example, in some embodiments, a C-8 on-line SPE column (such as an Oasis HLB on-line SPE column/cartridge (2.1 mm×20 mm) from Phenomenex, Inc. or equivalent) may be used to enrich adiponectin prior to mass spectrometric analysis. In some embodiments, use of an SPE column is conducted with HPLC Grade 0.2% aqueous formic acid as a wash solution, and use of 0.2% formic acid in acetonitrile as an elution solution.

In other embodiments, the methods include immunopurifying adiponectin prior to mass spectrometry analysis. The immunopurification step may be performed using any of the immunopurification methods well known in the art. Often the immunopurification procedure utilizes antibodies bound, conjugated, immobilized or otherwise attached to a solid support, for example a column, well, tube, capsule, particle or the like. Generally, immunopurification methods involve (1) incubating a sample containing the analyte of interest with antibodies such that the analyte binds to the antibodies, (2) performing one or more washing steps, and (3) eluting the analyte from the antibodies.

In some embodiments the incubation step of the immunopurification is performed with the antibodies free in solution and the antibodies are subsequently bound or attached to a solid surface prior to the washing steps. In some embodiments this can be achieved using a primary antibody that is an anti-adiponectin antibody and a secondary antibody attached to a solid surface that has an affinity to the primary anti-adiponectin antibody. In alternative embodiments, the primary antibody is bound to the solid surface prior to the incubation step.

Appropriate solid supports include without limitation tubes, slides, columns, beads, capsules, particles, gels, and the like. In some preferred embodiments, the solid support is a multi-well plate, such as, for example, a 96 well plate, a 384-well plate or the like. In some embodiments the solid support are sepharose or agarose beads or gels. There are numerous methods well known in the art by which antibodies (for example, an adiponectin antibody or a secondary antibody) may be bound, attached, immobilized or coupled to a solid support, e.g., covalent or non-covalent linkages adsorption, affinity binding, ionic linkages and the like. In some embodiments antibodies are coupled using CNBr, for example the antibodies may be coupled to CNBr activated sepharose. In other embodiments, the antibody is attached to the solid support through an antibody binding protein such as protein A, protein G, protein A/G, or protein L.

The washing step of the immunopurification methods generally involve washing the solid support such that the adiponectin remain bound to the anti-adiponectin antibodies on the solid support. The elution step of the immunopurification generally involves the addition of a solution that disrupts the binding of adiponectin to the anti-adiponectin antibodies. Exemplary elution solutions include organic solutions, salt solutions, and high or low pH solutions.

Another method of sample purification that may be used prior to mass spectrometry is liquid chromatography (LC). In liquid chromatography techniques, an analyte may be purified by applying a sample to a chromatographic analytical column under mobile phase conditions where the analyte of interest elutes at a differential rate in comparison to one or more other materials. Such procedures may enrich the amount of one or more analytes of interest relative to one or more other components of the sample.

Certain methods of liquid chromatography, including HPLC, rely on relatively slow, laminar flow technology. Traditional HPLC analysis relies on column packing in which laminar flow of the sample through the column is the basis for separation of the analyte of interest from the sample. The skilled artisan will understand that separation in such columns is a partition process and may select LC, including HPLC, instruments and columns that are suitable for use with adiponectin. The chromatographic analytical column typically includes a medium (i.e., a packing material) to facilitate separation of chemical moieties (i.e., fractionation). The medium may include minute particles. The particles typically include a bonded surface that interacts with the various chemical moieties to facilitate separation of the chemical moieties. One suitable bonded surface is a hydrophobic bonded surface such as an alkyl bonded or a cyano bonded surface. Alkyl bonded surfaces may include C-4, C-8, C-12, or C-18 bonded alkyl groups. In some embodiments, the chromatographic analytical column is a monolithic C-18 column. The chromatographic analytical column includes an inlet port for receiving a sample and an outlet port for discharging an effluent that includes the fractionated sample. The sample may be supplied to the inlet port directly, or from a SPE column, such as an on-line SPE column or a TFLC column. In some embodiments, an on-line filter may be used ahead of the SPE column and or HPLC column to remove particulates and phospholipids in the samples prior to the samples reaching the SPE and/or TFLC and/or HPLC columns.

In one embodiment, the sample may be applied to the LC column at the inlet port, eluted with a solvent or solvent mixture, and discharged at the outlet port. Different solvent modes may be selected for eluting the analyte(s) of interest. For example, liquid chromatography may be performed using a gradient mode, an isocratic mode, or a polytypic (i.e. mixed) mode. During chromatography, the separation of materials is effected by variables such as choice of eluent (also known as a “mobile phase”), elution mode, gradient conditions, temperature, etc.

The adiponectin in a sample may be purified by HPLC. This HPLC may be conducted with a monolithic C-18 column chromatographic system, for example, an Onyx Monolithic C-18 column from Phenomenex Inc. (50×2.0 mm), or equivalent. In certain embodiments, HPLC is performed using HPLC Grade 0.2% aqueous formic acid as solvent A, and 0.2% formic acid in acetonitrile as solvent B.

By appropriate selection of valves and connector plumbing, two or more chromatography columns may be connected as needed such that material is passed from one to the next without the need for any manual steps. The selection of valves and plumbing may be controlled by a computer pre-programmed to perform the necessary steps. The chromatography system may also be connected in an on-line fashion to the detector system, e.g., a mass spectroscopy system. Thus, an operator may place a tray of samples in an autosampler, and the remaining operations are performed under computer control, resulting in purification and analysis of all samples selected.

In some embodiments, TFLC may be used for purification of adiponectin prior to mass spectrometry. In such embodiments, samples may be extracted using a TFLC column which captures the analyte. The analyte is then eluted and transferred on-line to an analytical HPLC column. For example, sample extraction may be accomplished with a TFLC extraction cartridge with a large particle size (50 μm) packing. Sample eluted off of this column may then be transferred on-line to an HPLC analytical column for further purification prior to mass spectrometry. Because the steps involved in these chromatography procedures may be linked in an automated fashion, the requirement for operator involvement during the purification of the analyte can be minimized. This feature may result in savings of time and costs, and eliminate the opportunity for operator error.

In some embodiments, one or more of the above purification techniques may be used in parallel for purification of adiponectin to allow for simultaneous processing of multiple samples. In some embodiments, the purification techniques employed exclude immunopurification techniques, such as immunoaffinity chromatography.

### Detection and Quantitation of Adiponectin by Mass Spectrometry

Mass spectrometry is performed using a mass spectrometer, which includes an ion source for ionizing the fractionated sample and creating charged molecules for further analysis. In various embodiments, adiponectin may be ionized by any method known to the skilled artisan. For example, ionization of adiponectin may be performed by electron ionization, chemical ionization, electrospray ionization (ESI), photon ionization, atmospheric pressure chemical ionization (APCI), photoionization, atmospheric pressure photoionization (APPI), Laser diode thermal desorption (LDTD), fast atom bombardment (FAB), liquid secondary ionization (LSI), matrix assisted laser desorption ionization (MALDI), field ionization, field desorption, thermospray/plasmaspray ionization, surface enhanced laser desorption ionization (SELDI), inductively coupled plasma (ICP) and particle beam ionization. Based on the analyte to be measured, type of sample, the type of detector, the choice of positive versus negative mode, etc., the ionization method may be determined. Adiponectin may be ionized in positive or negative mode. In some embodiments, adiponectin is ionized by ESI in positive ion mode.

In mass spectrometry techniques generally, after the sample has been ionized, the positively or negatively charged ions thereby created may be analyzed to determine a mass to charge ratio (m/z). Various analyzers for determining m/z include quadrupole analyzers, ion traps analyzers, time-of-flight analyzers, Fourier transform ion cyclotron resonance mass analyzers, and orbitrap analyzers. Some exemplary ion trap methods are described in Bartolucci, et al., Rapid Commun. Mass Spectrom. 2000, 14:967-73.

The ions may be detected using several detection modes. For example, selected ions may be detected, i.e. using a selective ion monitoring mode (SIM), or alternatively, mass transitions resulting from collision induced dissociation or neutral loss may be monitored, e.g., multiple reaction monitoring (MRM) or selected reaction monitoring (SRM). In some embodiments, the mass-to-charge ratio is determined using a quadrupole analyzer. In a “quadrupole” or “quadrupole ion trap” instrument, ions in an oscillating radio frequency field experience a force proportional to the DC potential applied between electrodes, the amplitude of the RF signal, and the mass/charge ratio. The voltage and amplitude may be selected so that only ions having a particular mass/charge ratio travel the length of the quadrupole, while all other ions are deflected. Thus, quadrupole instruments may act as both a “mass filter” and as a “mass detector” for the ions injected into the instrument.

As ions collide with the detector they produce a pulse of electrons that are converted to a digital signal. The acquired data is relayed to a computer, which plots counts of the ions collected versus time. The resulting mass chromatograms are similar to chromatograms generated in traditional HPLC-MS methods. The areas under the peaks corresponding to particular ions, or the amplitude of such peaks, may be measured and correlated to the amount of the analyte of interest. In certain embodiments, the area under the curves, or amplitude of the peaks, for fragment ion(s) and/or precursor ions are measured to determine the amount of adiponectin. The relative abundance of a given ion may be converted into an absolute amount of the original analyte using calibration standard curves based on peaks of one or more ions of an internal or external molecular standard.

One may enhance the resolution of MS techniques employing certain mass spectrometric analyzers through “tandem mass spectrometry,” or “MS/MS”. In this technique, a precursor ion (also called a parent ion) generated from a molecule of interest can be filtered in an MS instrument, and the precursor ion subsequently fragmented to yield one or more fragment ions (also called daughter ions or product ions) that are then analyzed in a second MS procedure. By careful selection of precursor ions, only ions produced by certain analytes are passed to the fragmentation chamber, where collisions with atoms of an inert gas produce the fragment ions. Because both the precursor and fragment ions are produced in a reproducible fashion under a given set of ionization/fragmentation conditions, the MS/MS technique may provide an extremely powerful analytical tool. For example, the combination of filtration/fragmentation may be used to eliminate interfering substances, and may be particularly useful in complex samples, such as biological samples. In certain embodiments, a mass spectrometric instrument with multiple quadrupole analyzers (such as a triple quadrupole instrument) is employed to conduct tandem mass spectrometric analysis.

In certain embodiments using a MS/MS technique, precursor ions are isolated for further fragmentation, and collision activated dissociation (CAD) is used to generate fragment ions from the precursor ions for further detection. In CAD, precursor ions gain energy through collisions with an inert gas, and subsequently fragment by a process referred to as “unimolecular decomposition.” Sufficient energy must be deposited in the precursor ion so that certain bonds within the ion can be broken due to increased vibrational energy.

In some embodiments, adiponectin in a sample is detected and/or quantified using MS/MS as follows. Adiponectin is enriched in a sample by first subjecting the sample to SPE, then to liquid chromatography, preferably HPLC; the flow of liquid solvent from a chromatographic analytical column enters the heated nebulizer interface of an MS/MS analyzer; and the solvent/analyte mixture is converted to vapor in the heated charged tubing of the interface. During these processes, the analyte (i.e., adiponectin) is ionized. The ions, e.g. precursor ions, pass through the orifice of the instrument and enter the first quadrupole. Quadrupoles 1 and 3 (Q1 and Q3) are mass filters, allowing selection of ions (i.e., selection of “precursor” and “fragment” ions in Q1 and Q3, respectively) based on their mass to charge ratio (m/z). Quadrupole 2 (Q2) is the collision cell, where ions are fragmented. The first quadrupole of the mass spectrometer (Q1) selects for molecules with the m/z of an adiponectin ion. Precursor ions with the correct m/z are allowed to pass into the collision chamber (Q2), while unwanted ions with any other m/z collide with the sides of the quadrupole and are eliminated. Precursor ions entering Q2 collide with neutral gas molecules (such as Argon molecules) and fragment. The fragment ions generated are passed into quadrupole 3 (Q3), where the fragment ions are selected for detection.

Ionization of adiponectin may result in multiply charged precursor ions (such as precursor ions of 4+, 5+, 6+, etc.). Ionization conditions, particularly the pH of the buffer utilized in electrospray techniques, greatly influence the identity and quantity of adiponectin precursor ions generated. The methods may utilize either acidic or basic conditions; preferably acidic conditions.

The methods may involve MS/MS performed in either positive or negative ion mode; preferably positive ion mode. In certain embodiments, the electrospray buffer is acidic and Q1 selects for adiponectin precursor ions with an m/z of about 421.23±0.5. Fragmentation of either of these adiponectin precursor ions generates fragment ions with m/z of about 637.37±0.5, 540.31±0.5, and/or 319.19±0.5. Thus, in embodiments where Q1 selects for one or more adiponectin precursor ions selected from the group consisting of ions with m/z of about 421.23±0.5, Q3 may select one or more fragment ions selected from the group of ions with m/z of about 637.37±0.5, 540.31±0.5, and/or 319.19±0.5. In certain embodiments, the relative abundance of a single fragment ion from a single precursor ion may be measured. Alternatively, the relative abundances of two or more fragment ions from a single precursor ion may be measured. In these embodiments, the relative abundances of each fragment ion may be subjected to any known mathematical treatment to quantitatively assess adiponectin originally in the sample. In other embodiments, one or more fragment ions from two or more precursor ions may be measured and utilized as above to qualitatively assess adiponectin originally in the sample.

Alternate modes of operating a tandem mass spectrometric instrument that may be used in certain embodiments include product ion scanning and precursor ion scanning. For a description of these modes of operation, see, e.g., E. Michael Thurman, et al., Chromatographic-Mass Spectrometric Food Analysis for Trace Determination of Pesticide Residues, Chapter 8 (Amadeo R. Fernandez-Alba, ed., Elsevier 2005) (387).

In other embodiments, a high resolution/high accuracy mass analyzer may be used for quantitative analysis of adiponectin according to methods of the present invention. To achieve acceptable precision for quantitative results, the mass spectrometer must be capable of exhibiting a resolving power (FWHM) of 10,000 or more, with accuracy of about 50 ppm or less for the ions of interest; preferably the mass spectrometer exhibits a resolving power (FWHM) of 18,000 or better, with accuracy of about 5 ppm or less; such as a resolving power (FWHM) of 20,000 or better and accuracy of about 3 ppm or less; such as a resolving power (FWHM) of 25,000 or better and accuracy of about 3 ppm or less. Three exemplary analyzers capable of exhibiting the requisite level of performance for adiponectin ions are orbitrap mass analyzers, certain TOF mass analyzers, and Fourier transform ion cyclotron resonance mass analyzers.

Elements found in biological active molecules, such as carbon, oxygen, and nitrogen, naturally exist in a number of different isotopic forms. For example, most carbon is present as 12 C, but approximately 1% of all naturally occurring carbon is present as 13 C. Thus, some fraction of naturally occurring molecules containing at least one carbon atom will contain at least one 13 C atom. Inclusion of naturally occurring elemental isotopes in molecules gives rise to multiple molecular isotopic forms. The difference in masses of molecular isotopic forms is at least 1 atomic mass unit (amu). This is because elemental isotopes differ by at least one neutron (mass of one neutron≈1 amu). When molecular isotopic forms are ionized to multiply charged states, the mass distinction between the isotopic forms can become difficult to discern because mass spectrometric detection is based on the mass to charge ratio (m/z). For example, two isotopic forms differing in mass by 1 amu that are both ionized to a 5+ state will exhibit differences in their m/z of only 0.2. High resolution/high accuracy mass spectrometers are capable of discerning between isotopic forms of highly multiply charged ions (such as ions with charges of ±2, ±3, ±4, ±5, or higher).

Due to naturally occurring elemental isotopes, multiple isotopic forms typically exist for every molecular ion (each of which may give rise to a separately detectable spectrometric peak if analyzed with a sensitive enough mass spectrometric instrument). The m/z ratios and relative abundances of multiple isotopic forms collectively comprise an isotopic signature for a molecular ion. In some embodiments, the m/z ratios and relative abundances for two or more molecular isotopic forms may be utilized to confirm the identity of a molecular ion under investigation. In some embodiments, the mass spectrometric peak from one or more isotopic forms is used to quantitate a molecular ion. In some related embodiments, a single mass spectrometric peak from one isotopic form is used to quantitate a molecular ion. In other related embodiments, a plurality of isotopic peaks are used to quantitate a molecular ion. In these later embodiments, the plurality of isotopic peaks may be subject to any appropriate mathematical treatment. Several mathematical treatments are known in the art and include, but are not limited to summing the area under multiple peaks, or averaging the response from multiple peaks.

In some embodiments, the relative abundance of one or more ion is measured with a high resolution/high accuracy mass spectrometer in order to qualitatively assess the amount of adiponectin in the sample. In some embodiments, the one or more ions measured by high resolution/high accuracy mass spectrometry are multiply charged adiponectin ions.

Use of high resolution orbitrap analyzers has been reported for qualitative and quantitative analyses of various analytes. See, e.g., U.S. Patent Application Pub. No. 2008/0118932 (filed Nov. 9, 2007); Bredehoft, et al., Rapid Commun. Mass Spectrom., 2008, 22:477-485; Le Breton, et al., Rapid Commun. Mass Spectrom., 2008, 22:3130-36; Thevis, et al., Mass Spectrom. Reviews, 2008, 27:35-50; Thomas, et al., J. Mass Spectrom., 2008, 43:908-15; Schenk, et al., BMC Medical Genomics, 2008, 1:41; and Olsen, et al., Nature Methods, 2007, 4:709-12.

The results of an analyte assay may be related to the amount of the analyte in the original sample by numerous methods known in the art. For example, given that sampling and analysis parameters are carefully controlled, the relative abundance of a given ion may be compared to a table that converts that relative abundance to an absolute amount of the original molecule. Alternatively, external standards may be run with the samples, and a standard curve constructed based on ions generated from those standards. Using such a standard curve, the relative abundance of a given ion may be converted into an absolute amount of the original molecule. In certain preferred embodiments, an internal standard is used to generate a standard curve for calculating the quantity of adiponectin. Methods of generating and using such standard curves are well known in the art and one of ordinary skill is capable of selecting an appropriate internal standard. For example, in preferred embodiments one or more forms of isotopically labeled adiponectin may be used as internal standards. Numerous other methods for relating the amount of an ion to the amount of the original molecule will be well known to those of ordinary skill in the art.

As used herein, an “isotopic label” produces a mass shift in the labeled molecule relative to the unlabeled molecule when analyzed by mass spectrometric techniques. Examples of suitable labels include deuterium (2H), 13C, and 15N. One or more isotopic labels can be incorporated at one or more positions in the molecule and one or more kinds of isotopic labels can be used on the same isotopically labeled molecule.

In other embodiments, adiponectin may be subjected to a chemical treatment prior to mass spectrometric analysis. For example, adiponectin may be treated with TCEP (tris (2-carboxyethyl) phosphine).

The adiponectin may then be subject to any one or more of the purification steps described above for purification of adiponectin. In preferred embodiments, adiponectin is subject to purification by HPLC prior to mass spectrometric analysis.

Once purified, adiponectin is then subjected to an ionization source.

In preferred embodiments, adiponectin is ionized by ESI in positive mode.

One or more steps of any of the above described methods may be performed using automated machines. In certain embodiments, one or more purification steps are performed on-line, and more preferably all of the purification and mass spectrometry steps may be performed in an on-line fashion.

The following Examples serve to illustrate the invention. These Examples are in no way intended to limit the scope of the methods.

### EXAMPLES

### Example 1

Adiponectin LC/MS. General methods for quantification of adiponectin in a human serum sample via LC/MS. Calibration standards, quality controls (QC), and internal standards (IS) were made by reconstituting recombinant human adiponectin or peptides in MilliQ water and further diluting in stripped serum. Concentrations of calibrator and IS stock solutions were established using amino acid analysis (AAA) by an independent laboratory. Calibration standards, QCs, and patient specimens were thermally denatured and digested with TPCK (N-tosyl-L-phenylalanine chloromethyl ketone) trypsin after addition of IS in sodium deoxycholate (DOC) solution. Digestion was halted and DOC precipitated with formic acid. After centrifugation, sample supernatants were transferred to a 96-well plate for injection onto an Agilent StreamSelect LC system coupled to an Agilent 6495C QqQ MS. Adiponectin was chromatographically resolved using Kinetex 2.6 μm C18 columns (Phenomenex), and gradients of water and acetonitrile (both containing 0.1% formic acid). Analyte and IS were monitored using electrospray ionization in positive-ion mode with multiple reaction monitoring (MRM). The following ions were used in the analysis of adiponectin in this method.

| Compound | Adiponectin | Internal Standard |
|---|---|---|
| Ions (m/z) | 421.23 ± 0.5 | 416.22 ± 0.5 |
| | 314.18 ± 0.5 | 319.19 ± 0.5 |
| | 627.36 ± 0.5 | 637.37 ± 0.5 |
| | 530.30 ± 0.5 | 540.31 ± 0.5 |

### Example 2

Accuracy. A total of 98 samples were compared across 5 runs performed on 5 different days between a validated ELISA (enzyme-linked immunosorbent assay) method and MS Starscream (LC-MS/MS) under validation. The regression was evaluated for accuracy at the highest (37 μg/mL) and lowest (2 μg/mL) extremes of the population reference range.

The comparison between the validated ELISA method and LC/MS method yielded a slope of 1.008 with an intercept of −0.167 (Table 1; FIG. 1). The cut-points of 2, 15, and 37 μg/mL meet acceptance criteria with an average bias≤TEa/4. The bias for the comparison of samples between the validated ELISA method and MS Starscream is acceptable (within TEa/4).

TABLE 1
Comparison between the validated ELISA method and LC/MS method
Tea (Total Allowable Error): 30.0% or 1.50 μg/mL

| | | | | Difference, |
|---|---|---|---|---|
| Sample | | Mass Spec | Difference, | (Y − X)/X |
| # | ELISA (X) | (Y) | Y − X | (%) |
| 1 | 21.05 | 23.52 | 2.47 | 12% |
| 2 | 8.61 | 8.75 | 0.15 | 2% |
| 3 | 27.16 | 30.39 | 3.23 | 12% |
| 4 | 28.08 | 32.66 | 4.58 | 16% |
| 5 | 24.98 | 23.72 | −1.26 | −5% |
| 6 | 16.55 | 21.26 | 4.71 | 28% |
| 7 | 13.43 | 17.56 | 4.13 | 31% |
| 8 | 12.03 | 10.91 | −1.12 | −9% |
| 9 | 21.43 | 16.99 | −4.44 | −21% |
| 10 | 39.81 | 32.57 | −7.24 | −18% |
| 11 | 18.46 | 21.62 | 3.16 | 17% |
| 12 | 5.98 | 6.31 | 0.33 | 6% |
| 13 | 22.42 | 36.56 | 14.14 | 63% |
| 14 | 3.99 | 5.23 | 1.24 | 31% |
| 15 | 30.36 | 24.85 | −5.51 | −18% |
| 16 | 10.81 | 13.29 | 2.48 | 23% |
| 17 | 13.46 | 13.33 | −0.13 | −1% |
| 18 | 23.60 | 20.28 | −3.32 | −14% |
| 19 | 27.96 | 24.13 | −3.83 | −14% |
| 20 | 17.73 | 17.16 | −0.57 | −3% |
| 21 | 9.62 | 12.90 | 3.28 | 34% |
| 22 | 25.80 | 26.76 | 0.96 | 4% |
| 23 | 11.83 | 7.46 | −4.37 | −37% |
| 24 | 17.81 | 22.25 | 4.44 | 25% |
| 25 | 11.51 | 8.42 | −3.09 | −27% |
| 26 | 17.30 | 12.81 | −4.49 | −26% |
| 27 | 15.41 | 11.54 | −3.87 | −25% |
| 28 | 13.90 | 20.13 | 6.23 | 45% |
| 29 | 28.66 | 28.91 | 0.25 | 1% |
| 30 | 8.93 | 11.35 | 2.42 | 27% |
| 31 | 19.89 | 35.33 | 15.45 | 78% |
| 32 | 14.63 | 10.80 | −3.83 | −26% |
| 33 | 31.57 | 32.98 | 1.41 | 4% |
| 34 | 10.86 | 10.42 | −0.44 | −4% |
| 35 | 14.60 | 8.85 | −5.75 | −39% |
| 36 | 13.09 | 14.08 | 0.99 | 8% |
| 37 | 12.23 | 7.47 | −4.76 | −39% |
| 38 | 23.03 | 19.03 | −4.00 | −17% |
| 39 | 8.23 | 16.37 | 8.14 | 99% |
| 40 | 22.63 | 18.95 | −3.68 | −16% |
| 41 | 3.56 | 5.76 | 2.20 | 62% |
| 42 | 17.99 | 21.19 | 3.20 | 18% |
| 43 | 18.36 | 25.44 | 7.08 | 39% |
| 44 | 28.00 | 31.62 | 3.62 | 13% |
| 45 | 33.72 | 24.10 | −9.62 | −29% |
| 46 | 26.43 | 22.47 | −3.96 | −15% |
| 47 | 31.40 | 23.05 | −8.35 | −27% |
| 48 | 16.02 | 15.38 | −0.64 | −4% |
| 49 | 7.22 | 3.22 | −4.00 | −55% |
| 50 | 22.66 | 18.61 | −4.05 | −18% |
| 51 | 23.53 | 31.12 | 7.59 | 32% |
| 52 | 40.00 | 27.59 | −12.41 | −31% |
| 53 | 40.00 | 34.05 | −5.95 | −15% |
| 54 | 40.00 | 29.65 | −10.35 | −26% |
| 55 | 8.60 | 8.80 | 0.20 | 2% |
| 56 | 8.60 | 10.20 | 1.60 | 19% |
| 57 | 8.60 | 8.47 | −0.13 | −2% |
| 58 | 7.70 | 10.10 | 2.40 | 31% |
| 59 | 6.40 | 5.00 | −1.40 | −22% |
| 60 | 12.65 | 9.27 | −3.38 | −27% |
| 61 | 14.38 | 12.31 | −2.07 | −14% |
| 62 | 10.55 | 7.98 | −2.57 | −24% |
| 63 | 23.53 | 21.47 | −2.06 | −9% |
| 64 | 24.41 | 30.40 | 5.99 | 25% |
| 65 | 20.65 | 18.56 | −2.09 | −10% |
| 66 | 15.72 | 20.24 | 4.52 | 29% |
| 67 | 35.40 | 35.83 | 0.43 | 1% |
| 68 | 5.20 | 4.67 | −0.53 | −10% |
| 69 | 35.40 | 40.69 | 5.29 | 15% |
| 70 | 5.20 | 4.79 | −0.41 | −8% |
| 71 | 17.10 | 18.67 | 1.57 | 9% |
| 72 | 17.10 | 18.51 | 1.41 | 8% |
| 73 | 17.10 | 16.52 | −0.58 | −3% |
| 74 | 17.10 | 20.25 | 3.15 | 18% |
| 75 | 17.10 | 18.47 | 1.37 | 8% |
| 76 | 40.00 | 39.31 | −0.69 | −2% |
| 77 | 40.00 | 40.49 | 0.49 | 1% |
| 78 | 40.00 | 39.58 | −0.42 | −1% |
| 79 | 40.00 | 37.98 | −2.02 | −5% |
| 80 | 40.00 | 40.23 | 0.23 | 1% |
| 81 | 1.56 | 1.68 | 0.12 | 8% |
| 82 | 3.13 | 2.45 | −0.68 | −22% |
| 83 | 6.25 | 6.26 | 0.01 | 0% |
| 84 | 12.50 | 9.83 | −2.67 | −21% |
| 85 | 25.00 | 29.42 | 4.42 | 18% |
| 86* | 50.00 | 50.80 | 0.80 | 2% |
| 87 | 17.10 | 18.97 | 1.87 | 11% |
| 88 | 17.10 | 17.62 | 0.52 | 3% |
| 89 | 17.10 | 18.74 | 1.64 | 10% |
| 90 | 17.10 | 17.44 | 0.34 | 2% |
| 91 | 17.10 | 17.01 | −0.09 | −1% |
| 92 | 17.10 | 17.12 | 0.02 | 0% |
| 93 | 8.60 | 8.97 | 0.37 | 4% |
| 94 | 8.60 | 8.96 | 0.36 | 4% |
| 95 | 8.60 | 8.75 | 0.15 | 2% |
| 96 | 8.60 | 9.09 | 0.49 | 6% |
| 97 | 8.60 | 9.51 | 0.91 | 11% |
| 98 | 8.60 | 9.16 | 0.56 | 7% |

*In Sample 86, a value above the highest calibration standard (above the analytical measurement range) was recovered.

### Example 3

Inter-Day Precision. QC pools containing high, medium, and low concentrations of recombinant human adiponectin (QCH, QCM, QCL) were prepared in bulk and stored as single use aliquots in −80° C. For intra-assay precision, 20 replicates of each level were prepared and analyzed on the same day in the same batch. For inter-assay precision, 5 replicates of each level were evaluated over 20 separate runs.

QC low material yielded a Grand Mean of 4.01 μg/mL with an average within-run SD (Standard Deviation) of 0.38 μg/mL (within-run CV (Coefficient of Variation)=9.40%) and a total SD of 0.40 μg/mL (total CV=9.94%) for a Sigma Overall=3.76. Two samples highlighted in yellow and bolded were removed from data calculations as were deemed as outliers.

QC medium material yielded a Grand Mean of 13.28 μg/mL with an average within-run SD of 0.83 μg/mL (within-run CV=6.28%) and a total SD of 0.92 μg/mL (total CV=6.90%) for a Sigma Overall=4.35.

QC High material yielded a Grand Mean of 22.30 μg/mL with an average within-run SD of 1.29 μg/mL (within-run CV=5.78%) and a total SD of 1.43 μg/mL (total CV=6.40%) for a Sigma Overall=4.69.

All three levels of QC meet total precision acceptance criteria of TEa/3 (Table 2; FIG. 2).

TABLE 2
Inter-Day Precision and Levels of QC

| QC | | | | | | | | w/i Run |
|---|---|---|---|---|---|---|---|---|
| Low | Rep 1 | Rep 2 | Rep 3 | Rep 4 | Rep 5 | Count | Average | SD |
| 1 | 4.08 | 4.21 | 3.89 | 4.15 | 3.63 | 5 | 3.99 | 0.24 |
| 2 | 3.78 | 4.17 | 3.56 | 2.34 | 4.13 | 4 | 3.91 | 0.29 |
| 3 | 3.70 | 4.28 | 4.30 | 4.21 | 4.53 | 5 | 4.20 | 0.31 |
| 4 | 4.01 | 4.95 | 3.93 | 4.26 | 4.73 | 5 | 4.38 | 0.45 |
| 5 | 4.13 | 5.94 | 4.40 | 3.87 | 4.13 | 4 | 4.13 | 0.22 |
| 6 | 3.49 | 4.13 | 3.53 | 3.75 | 4.15 | 5 | 3.81 | 0.32 |
| 7 | 3.84 | 3.22 | 3.71 | 3.63 | 3.98 | 5 | 3.68 | 0.29 |
| 8 | 3.27 | 4.27 | 4.04 | 4.09 | 4.01 | 5 | 3.94 | 0.39 |
| 9 | 4.38 | 3.37 | 4.15 | 3.38 | 3.11 | 5 | 3.68 | 0.55 |
| 10 | 4.28 | 3.89 | 4.13 | 4.14 | 4.34 | 5 | 4.16 | 0.17 |
| 11 | 3.90 | 4.24 | 3.71 | 3.98 | 3.92 | 5 | 3.95 | 0.19 |
| 12 | 3.83 | 3.92 | 3.95 | 3.87 | 3.53 | 5 | 3.82 | 0.17 |
| 13 | 3.88 | 4.03 | 4.25 | 4.47 | 4.26 | 5 | 4.18 | 0.23 |
| 14 | 4.50 | 3.36 | 4.62 | 4.55 | 4.35 | 5 | 4.28 | 0.52 |
| 15 | 4.38 | 4.57 | 3.46 | 3.62 | 3.89 | 5 | 3.98 | 0.48 |
| 16 | 4.57 | 3.36 | 4.20 | 3.58 | 4.30 | 5 | 4.00 | 0.51 |
| 17 | 4.11 | 4.35 | 3.75 | 3.55 | 4.72 | 5 | 4.10 | 0.47 |
| 18 | 3.73 | 3.58 | 4.21 | 3.75 | 3.65 | 5 | 3.78 | 0.25 |
| 19 | 4.38 | 4.55 | 4.29 | 4.30 | 3.57 | 5 | 4.22 | 0.38 |
| 20 | 3.47 | 4.41 | 5.09 | 4.72 | 4.29 | 5 | 4.40 | 0.60 |
| 21 | 13.49 | 13.40 | 13.85 | 13.18 | 14.93 | 5 | 13.77 | 0.69 |
| 22 | 14.39 | 13.77 | 12.28 | 13.97 | 13.37 | 5 | 13.56 | 0.80 |
| 23 | 13.73 | 13.18 | 12.33 | 13.09 | 13.63 | 5 | 13.19 | 0.56 |
| 24 | 13.98 | 13.88 | 15.24 | 15.37 | 14.31 | 5 | 14.56 | 0.70 |
| 25 | 12.14 | 10.91 | 12.20 | 14.14 | 12.31 | 5 | 12.34 | 1.16 |
| 26 | 13.27 | 13.29 | 14.18 | 13.79 | 13.32 | 5 | 13.57 | 0.40 |
| 27 | 12.76 | 13.00 | 12.58 | 13.89 | 13.77 | 5 | 13.20 | 0.60 |
| 28 | 14.28 | 13.29 | 13.92 | 13.10 | 14.06 | 5 | 13.73 | 0.51 |
| 29 | 11.92 | 12.73 | 12.93 | 13.17 | 10.55 | 5 | 12.26 | 1.07 |
| 30 | 12.34 | 13.57 | 13.84 | 13.36 | 13.96 | 5 | 13.41 | 0.64 |
| 31 | 12.00 | 12.38 | 13.43 | 14.57 | 14.69 | 5 | 13.41 | 1.23 |
| 32 | 11.62 | 12.92 | 12.64 | 12.57 | 12.61 | 5 | 12.47 | 0.50 |
| 33 | 14.33 | 13.07 | 13.39 | 13.88 | 13.14 | 5 | 13.56 | 0.53 |
| 34 | 13.08 | 12.67 | 12.26 | 13.36 | 12.41 | 5 | 12.76 | 0.46 |
| 35 | 15.11 | 13.27 | 12.91 | 13.13 | 14.50 | 5 | 13.78 | 0.97 |
| 36 | 12.81 | 13.73 | 11.90 | 14.31 | 13.12 | 5 | 13.17 | 0.92 |
| 37 | 12.69 | 11.96 | 12.90 | 14.71 | 14.53 | 5 | 13.36 | 1.21 |
| 38 | 13.46 | 13.51 | 13.44 | 12.60 | 13.27 | 5 | 13.26 | 0.38 |
| 39 | 13.74 | 13.85 | 12.80 | 12.03 | 13.23 | 5 | 13.13 | 0.74 |
| 40 | 12.89 | 10.98 | 13.83 | 13.34 | 14.98 | 5 | 13.20 | 1.47 |
| 41 | 22.88 | 22.06 | 23.75 | 22.28 | 21.18 | 5 | 22.43 | 0.96 |
| 42 | 21.44 | 23.26 | 22.95 | 21.92 | 22.03 | 5 | 22.32 | 0.76 |
| 43 | 25.51 | 22.35 | 22.61 | 22.61 | 20.06 | 5 | 22.63 | 1.94 |
| 44 | 20.23 | 21.93 | 20.96 | 20.07 | 20.07 | 5 | 20.65 | 0.80 |
| 45 | 23.78 | 24.62 | 22.78 | 20.74 | 21.72 | 5 | 22.73 | 1.55 |
| 46 | 22.42 | 22.96 | 22.81 | 23.24 | 24.18 | 5 | 23.12 | 0.66 |
| 47 | 22.71 | 22.99 | 22.77 | 20.92 | 23.99 | 5 | 22.68 | 1.11 |
| 48 | 21.43 | 23.00 | 23.10 | 21.14 | 23.18 | 5 | 22.37 | 1.00 |
| 49 | 21.98 | 22.23 | 22.96 | 22.20 | 23.03 | 5 | 22.48 | 0.48 |
| 50 | 22.35 | 23.08 | 21.53 | 20.23 | 22.27 | 5 | 21.89 | 1.08 |
| 51 | 22.88 | 21.74 | 20.38 | 19.33 | 20.72 | 5 | 21.01 | 1.35 |
| 52 | 23.07 | 23.65 | 24.12 | 22.15 | 22.43 | 5 | 23.08 | 0.82 |
| 53 | 22.28 | 23.80 | 23.29 | 23.50 | 24.08 | 5 | 23.39 | 0.69 |
| 54 | 22.40 | 22.84 | 24.65 | 23.66 | 21.13 | 5 | 22.94 | 1.32 |
| 55 | 20.70 | 20.19 | 24.46 | 22.53 | 24.80 | 5 | 22.54 | 2.10 |
| 56 | 21.01 | 23.76 | 21.32 | 20.34 | 19.23 | 5 | 21.13 | 1.67 |
| 57 | 21.35 | 25.77 | 21.79 | 19.08 | 21.73 | 5 | 21.94 | 2.41 |
| 58 | 22.84 | 23.50 | 22.41 | 22.14 | 22.18 | 5 | 22.61 | 0.57 |
| 59 | 22.95 | 24.60 | 21.41 | 24.28 | 24.19 | 5 | 23.49 | 1.32 |
| 60 | 20.04 | 19.59 | 21.41 | 21.97 | 20.13 | 5 | 20.63 | 1.01 |

TABLE 3
Summary

| Summary | Level 1 | Level 2 | Level 3 |
|---|---|---|---|
| Count | 98 | 100 | 100 |
| Grand Mean | 4.01 | 13.28 | 22.30 |
| Average within-run SD | 0.38 | 0.83 | 1.29 |
| Average within-run CV | 9.40% | 6.28% | 5.78% |
| Total SD | 0.40 | 0.92 | 1.43 |
| Total CV | 9.94% | 6.90% | 6.40% |
| Sigma Overall | 3.76 | 4.35 | 4.69 |
| Precision >= 3.0 sigma? | YES | YES | YES |

### Example 4: Intra-Day Precision

Level 1 (Low QC) yielded a mean of 4.53 μg/mL. The within-run SD was 0.37 μg/mL (with-in run CV=8.13%).
Level 2 (Mid QC) yielded a mean of 14.02 μg/mL. The within-run SD was 1.03 μg/mL (with-in run CV=7.37%).
Level 3 (High QC) yielded a mean of 24.08 μg/mL. The within-run SD was 1.56 μg/mL (with-in run CV=6.48%).

All three levels of QC meet total precision acceptance criteria of TEa/3 (Table 4).

TABLE 4
Intra-Day Precision and Levels of QC

| Level 1 | Rep 1 |
|---|---|
| 1 | 4.20 |
| 2 | 4.41 |
| 3 | 4.68 |
| 4 | 4.43 |
| 5 | 4.56 |
| 6 | 4.52 |
| 7 | 4.69 |
| 8 | 5.08 |
| 9 | 4.20 |
| 10 | 3.88 |
| 11 | 5.20 |
| 12 | 5.20 |
| 13 | 4.77 |
| 14 | 4.26 |
| 15 | 4.03 |
| 16 | 4.25 |
| 17 | 4.47 |
| 18 | 4.32 |
| 19 | 4.46 |
| 20 | 4.91 |

Level 1

| | |
|---|---|
| N | 20 |
| X | 4.53 |
| w/i Run SD | 0.37 |
| w/i Run CV | 8.13% |
| Sigma | 4.08 |
| Prec >3.0 | YES |

| Level 2 | Rep 1 |
|---|---|
| 1 | 13.57 |
| 2 | 15.29 |
| 3 | 13.88 |
| 4 | 13.14 |
| 5 | 13.21 |
| 6 | 12.67 |
| 7 | 14.76 |
| 8 | 13.06 |
| 9 | 15.31 |
| 10 | 14.26 |
| 11 | 15.91 |
| 12 | 12.99 |
| 13 | 15.09 |
| 14 | 12.59 |
| 15 | 14.33 |
| 16 | 13.86 |
| 17 | 14.33 |
| 18 | 13.07 |
| 19 | 13.39 |
| 20 | 15.66 |

Level 2

| | |
|---|---|
| N | 20 |
| X | 14.02 |
| w/i Run | 1.03 |
| SD | |
| w/i Run | 7.37% |
| CV | |
| Sigma | 4.07 |
| Prec >3.0 | YES |

| Level 3 | Rep 1 |
|---|---|
| 1 | 24.76 |
| 2 | 24.26 |
| 3 | 24.58 |
| 4 | 26.02 |
| 5 | 25.92 |
| 6 | 27.49 |
| 7 | 24.19 |
| 8 | 25.30 |
| 9 | 24.51 |
| 10 | 25.61 |
| 11 | 22.77 |
| 12 | 23.31 |
| 13 | 23.47 |
| 14 | 22.28 |
| 15 | 23.80 |
| 16 | 23.29 |
| 17 | 23.50 |
| 18 | 24.08 |
| 19 | 21.42 |
| 20 | 21.13 |

Level 3

| | |
|---|---|
| N | 20 |
| X | 24.08 |
| w/i Run | 1.56 |
| SD | |
| w/i Run | 6.48% |
| CV | |
| Sigma | 4.63 |
| Prec ≥3.0 | YES |

### Example 5

Analytical Measurement Range (AMR). AMR verification was performed by using two different lots of recombinant protein with target concentrations assigned by third-party amino acid analysis and serially diluted across the AMR. The lowest and highest value samples had target values within 1.5 TEa of the lower limit and upper limits of the AMR, respectively. All samples were measured in triplicate replicates.

The acceptance criteria for adiponectin is that the average of three replicate measurements at each level are recovered within TEa/4 (7.5% or 0.375 μg/mL), from 1.50 ug/mL to 50.00 μg/mL.

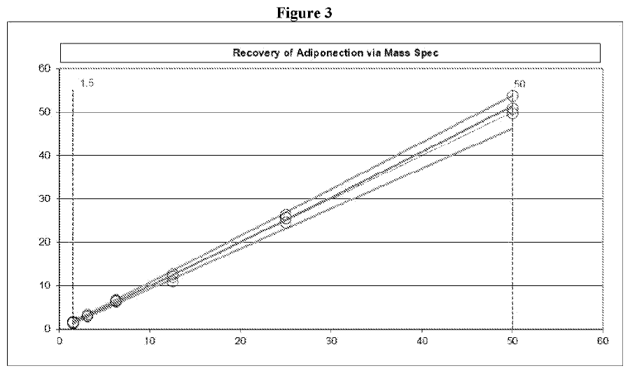

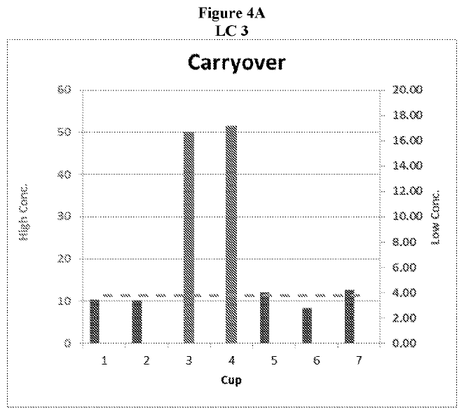

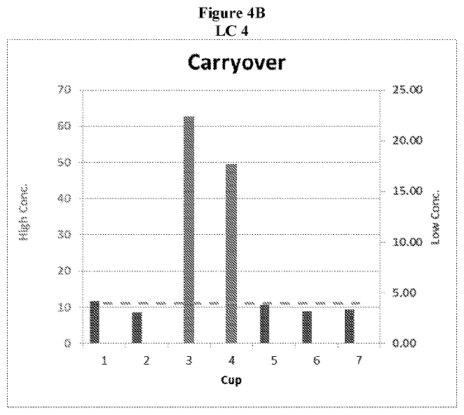

Acceptance criteria was met across the AMR from 1.50 μg/mL to 50.00 μg/mL (Table 5; FIG. 3).

TABLE 5
AMR and Acceptance Criteria
TEa: 30% or 1.50 μg/mL

| Sample | Target | Rep1 | Rep2 | Rep3 | Average | TEa/4 | min | max | w/i range | Index |
|---|---|---|---|---|---|---|---|---|---|---|
| 1 | 1.56 | 1.68 | 1.5 | 1.3 | 1.52 | 0.38 | 1.19 | 1.94 | YES | −12% |
| 2 | 3.13 | 3.11 | 3.0 | 3.4 | 3.16 | 0.38 | 2.75 | 3.50 | YES | 8% |
| 3 | 6.25 | 6.45 | 6.4 | 6.6 | 6.50 | 0.47 | 5.78 | 6.72 | YES | 53% |
| 4 | 12.5 | 10.9 | 12.1 | 12.7 | 11.89 | 0.94 | 11.56 | 13.44 | YES | −65% |
| 5 | 25.0 | 26.3 | 25.6 | 24.6 | 25.48 | 1.88 | 23.13 | 26.88 | YES | 26% |
| 6 | 50.0 | 50.8 | 53.7 | 49.7 | 51.39 | 3.75 | 46.25 | 53.75 | YES | 37% |

Analysis
y = 1.03 × −0.238

| | |
|---|---|
| LL of AMR bracketed | YES |
| UL of AMR bracketed | YES |
| LL recovered | YES |
| UL recovered | YES |
| Mid-point recovered | YES |
| AMR validated | YES |

### Example 6

Carryover. Samples were injected three separate times using the following sequence: low concentration, low concentration, high concentration, high concentration, low concentration, low concentration, low concentration (low concentration material was QC Low and high concentration material was calibrator 1).

Acceptance criteria is that carryover from the high concentration sample to a low concentration sample does not exceed TEa/4 for each reported parameter.

Carryover and pre-carryover did not exceed TEa/4 for adiponectin, thus passing acceptance criteria (Table 6, FIG. 4).

TABLE 6
Carryover.
LC3
TEa: 30.0% or 1.50 μg/mL

| Cup # | Type | Run #1 | Run #2 | Run #3 |
|---|---|---|---|---|
| 1 | Low | 3.730 | 3.190 | 3.340 |
| 2 | Low | 3.680 | 3.180 | 3.220 |
| 3 | High | 50.240 | 48.850 | 51.310 |
| 4 | High | 53.470 | 51.340 | 49.790 |
| 5 | Low | 3.990 | 3.850 | 4.290 |
| 6 | Low | 3.290 | 1.690 | 3.280 |
| 7 | Low | 4.320 | 3.900 | 4.410 |

| | | | |
|---|---|---|---|
| Average Low | 4.025 | 3.545 | 3.875 |
| Average High | 51.9 | 50.1 | 50.6 |
| Carry over in Cup 5 | −0.035 | 0.305 | 0.415 |
| Carry over in Cup 6 | −0.735 | −1.855 | −0.595 |
| Pre-Carry over in Cup 2 | −0.345 | −0.365 | −0.655 |

| | Average | TEa/4 | Carry over ? |
|---|---|---|---|
| Carry over in Cup 5 | 0.228 | 0.375 | No carry over |
| Carry over in Cup 6 | −1.062 | 0.375 | No carry over |
| Pre-Carry over in Cup 2 | −0.455 | 0.375 | No carry over |

LC4
TEa 30% or 1.50 μg/mL

| Cup # | Type | Run #1 | Run #2 | Run #3 |
|---|---|---|---|---|
| 1 | Low | 4.300 | 3.670 | 4.420 |
| 2 | Low | 3.310 | 3.010 | 2.720 |
| 3 | High | 62.760 | 59.040 | 66.280 |
| 4 | High | 50.400 | 49.290 | 48.820 |
| 5 | Low | 3.490 | 4.370 | 3.430 |
| 6 | Low | 4.230 | 1.900 | 3.390 |
| 7 | Low | 3.460 | 3.510 | 3.140 |

| | | | |
|---|---|---|---|
| Average Low | 3.880 | 3.590 | 3.780 |
| Average High | 56.6 | 54.2 | 57.6 |
| Carry over in Cup 5 | −0.390 | 0.780 | −0.350 |
| Carry over in Cup 6 | 0.350 | −1.690 | −0.390 |
| Pre Carry over in Cup 2 | −0.570 | −0.580 | −1.060 |

| | Average | TEa/4 | Carry over ? |
|---|---|---|---|
| Carry over in Cup 5 | 0.013 | 0.375 | No carry over |
| Carry over in Cup 6 | −0.577 | 0.375 | No carry over |
| Pre-Carry over in Cup 2 | −0.737 | 0.375 | No carry over |

### Example 7

Analytical Sensitivity. The limit of quantification (LOQ), limit of blank (LOB) and limit of detection (LOD) was verified by analyzing 25 replicate matrix blanks and 25 replicates at the lower limit of the AMR. Samples for lower limit testing were created by spiking stripped serum recombinant human adiponectin to obtain concentrations for each parameter.

Acceptance criteria is concentration of the low pool<(LL AMR+1.5*TEa) and imprecision of the low pool is SD<TEa/3.

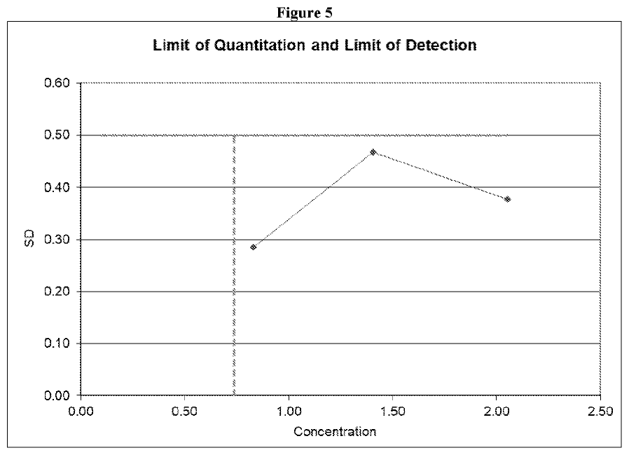

LOB was calculated to be 0.419 μg/mL, LOD of 0.740 μg/mL, and LOQ of 0.831 μg/mL. Each of the low pools had a SD<TEa/3 and passed acceptance criteria (Table 7, FIG. 5).

TABLE 7
Analytical Sensitivity
TEa 30% or 1.50 μg/mL

| Run # | Result # | Pool A | Pool B | Pool C | zero cal |
|---|---|---|---|---|---|
| 1 | 1 | 0.580 | 0.910 | 1.750 | 0.000 |
| | 2 | 0.480 | 0.960 | 2.190 | 0.000 |
| | 3 | 0.660 | 1.220 | 1.680 | 0.000 |
| | 4 | 1.200 | 0.970 | 2.850 | 0.420 |
| | 5 | 0.790 | 1.290 | 2.880 | 0.150 |
| 2 | 1 | 1.670 | 2.630 | 1.510 | 0.000 |
| | 2 | 0.920 | 0.910 | 2.120 | 0.000 |
| | 3 | 0.990 | 1.740 | 2.010 | 0.000 |
| | 4 | 1.010 | 1.560 | 2.320 | 0.230 |
| | 5 | 0.640 | 0.900 | 2.180 | 0.210 |
| 3 | 1 | 0.540 | 0.990 | 2.310 | 0.000 |
| | 2 | 0.820 | 0.970 | 1.870 | 0.000 |
| | 3 | 0.960 | 1.490 | 1.690 | 0.000 |
| | 4 | 0.820 | 1.540 | 2.150 | 0.000 |
| | 5 | 1.130 | 1.440 | 1.800 | 0.210 |
| 4 | 1 | 0.850 | 0.860 | 2.270 | 0.000 |
| | 2 | 1.280 | 1.080 | 2.390 | 0.000 |
| | 3 | 0.860 | 1.400 | 2.000 | 0.090 |
| | 4 | 0.830 | 1.890 | 1.450 | 0.560 |
| | 5 | 0.660 | 1.260 | 2.060 | 0.000 |
| 5 | 1 | 0.390 | 1.570 | 1.640 | 0.410 |
| | 2 | 0.580 | 2.290 | 2.110 | 0.010 |
| | 3 | 0.890 | 2.100 | 1.850 | 0.000 |
| | 4 | 0.680 | 1.510 | 1.660 | 0.170 |
| | 5 | 0.540 | 1.690 | 2.570 | 0.000 |

| | | | | |
|---|---|---|---|---|
| Count | 25 | 25 | 25 | 25 |
| Mean | 0.831 | 1.407 | 2.052 | 0.098 |
| SD | 0.285 | 0.468 | 0.378 | 0.160 |
| TEa/3 | 0.500 | 0.500 | 0.500 | |
| Is SD < TEa/3 ? | YES | YES | YES | |
| Is Mean >= LOD ? | YES | YES | YES | |

| | |
|---|---|
| Limit of Blank (LOB) | 0.419 |
| Limit of Detection (LOD) | 0.740 |
| Limit of Quantitation (LOQ) | 0.831 |

### Example 8

Sample Stability. Stability was evaluated by measuring adiponectin in a minimum of 7 freshly drawn single donor serum samples over time at ambient (18 to 25° C.), refrigerated (2 to 8° C.), frozen (−20° C.), and deep-frozen (−70° C.) temperature conditions.

The acceptance criteria is the average recovered value of evaluated samples not deviate from Day 0 by more than TEa/3 (the greater of 10% or 0.5 μg/mL).
Ambient Temperature (18 to 25° C.)

Samples were assayed at 0, 24, 48 and 96 hours.

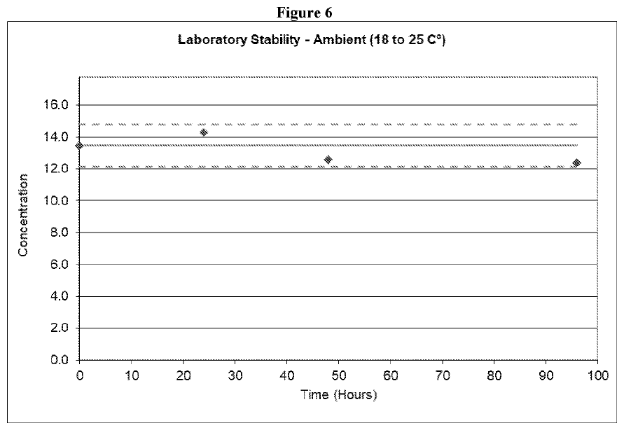

The average concentration was within TEa/3 of the average concentration measured at t=0 for all assessed timepoints. (Table 8; FIG. 6). Adiponectin is stable up to 96 hours at ambient temperature.

TABLE 8
Sample Stability (Ambient)
TEa: 30% or 1.50 μg/mL

| | |
|---|---|
| Ambient (18 to 25 C. °) | Hours |

| Sample # | 0 | 24 | 48 | 96 |
|---|---|---|---|---|
| 1 | 11.26 | 9.37 | 10.18 | 8.95 |
| 2 | 24.44 | 17.52 | 17.87 | 15.61 |
| 3 | 8.73 | 7.16 | 6.94 | 5.80 |
| 4 | 17.38 | 28.71 | 18.69 | 15.19 |
| 5 | 10.70 | 13.81 | 12.52 | 12.47 |
| 6 | 7.82 | 9.40 | 6.74 | 8.13 |
| 7 | 8.54 | 8.65 | 9.45 | 9.90 |
| 9 | 6.52 | 9.24 | 6.62 | 5.34 |
| 10 | 25.66 | 24.81 | 24.08 | 30.18 |
| Average | 13.45 | 14.30 | 12.57 | 12.40 |

| | |
|---|---|
| Target +TEa/3 | 14.80 |
| Day 0 Target | 13.45 |
| Target −TEa/3 | 12.11 |

Refrigerated (2 to 8° C.)

Samples were assayed at 0, 2, 4, 6 and 20 days.

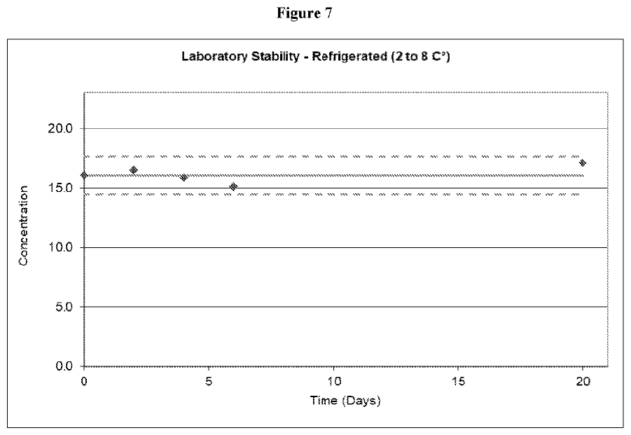

The average concentration was within TEa/3 of the average concentration measured at t=0 for all assessed timepoints (Table 9; FIG. 7). Adiponectin is stable up to 20 days at refrigerated temperature.

TABLE 9
Sample Stability (Refrigerated)
TEa: 30% or 1.50 μg/mL

| Refrigerated (2 to 8 C. °) | Days | | | | |
|---|---|---|---|---|---|
| Sample # | 0 | 2 | 4 | 6 | 20 |
| 1 | 11.26 | 10.17 | 12.12 | 10.13 | 10.96 |
| 2 | 24.44 | 15.09 | 17.62 | 16.47 | 22.27 |
| 3 | 8.73 | 5.82 | 8.09 | 7.47 | 10.23 |
| 4 | 17.38 | 14.46 | 18.04 | 17.20 | 15.25 |
| 5 | 10.70 | 14.95 | 13.43 | 13.55 | 17.27 |
| 6 | 7.82 | 5.73 | 8.29 | 7.55 | 9.85 |
| 7 | 8.54 | 16.77 | 8.53 | 8.99 | 12.84 |
| 8 | 39.77 | 40.91 | 40.86 | 35.21 | 39.90 |
| 9 | 6.52 | 7.03 | 7.30 | 7.06 | 5.73 |
| 10 | 25.66 | 34.27 | 24.66 | 27.45 | 27.19 |
| Average | 16.08 | 16.52 | 15.89 | 15.11 | 17.15 |

| | |
|---|---|
| Target +TEa/3 | 17.69 |
| Day 0 Target | 16.08 |
| Target −TEa/3 | 14.47 |

Frozen (−20° C.)

Samples were assayed at 0, 6, 13, 20 and 27 days.

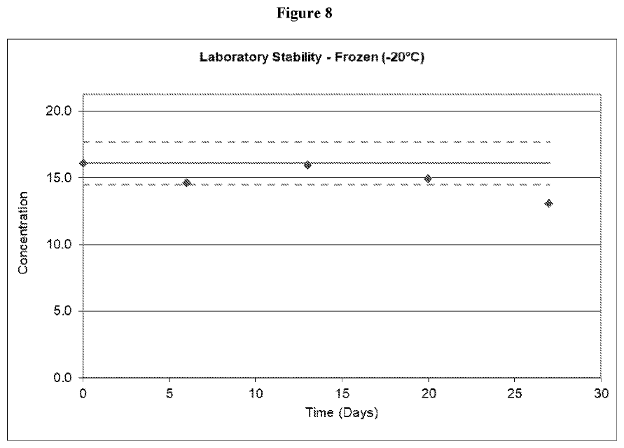

The average concentration was within TEa/3 of the average concentration measured at t=0 for all time points up to 20 days (Table 10; FIG. 8). Adiponectin is stable up to 20 days at frozen temperature.

TABLE 10
Sample Stability (Frozen)
TEa: 30% or 1.50 μg/mL

| | |
|---|---|
| Frozen (−20° C.) | Days |

| Sample # | 0 | 6 | 13 | 20 | 27 |
|---|---|---|---|---|---|
| 1 | 11.26 | 10.59 | 9.96 | 9.75 | 9.09 |
| 2 | 24.44 | 20.00 | 15.13 | 19.43 | 12.61 |
| 3 | 8.73 | 5.35 | 7.94 | 7.66 | 5.42 |
| 4 | 17.38 | 16.40 | 20.75 | 18.33 | 15.66 |
| 5 | 10.70 | 14.35 | 14.46 | 11.40 | 12.23 |
| 6 | 7.82 | 4.94 | 6.41 | 6.14 | 5.65 |
| 7 | 8.54 | 9.45 | 7.10 | 9.49 | 7.13 |
| 8 | 39.77 | 34.56 | 43.77 | 37.89 | 34.92 |
| 9 | 6.52 | 7.18 | 4.46 | 4.43 | 3.99 |
| 10 | 25.66 | 23.27 | 29.47 | 24.83 | 23.12 |
| Average | 16.08 | 14.61 | 15.95 | 14.94 | 12.98 |

| | |
|---|---|
| Target +TEa/3 | 17.69 |
| Day 0 Target | 16.08 |
| Target −TEa/3 | 14.47 |

Deep Frozen (<−70° C.)

Samples were assayed at 0 and 27 days.

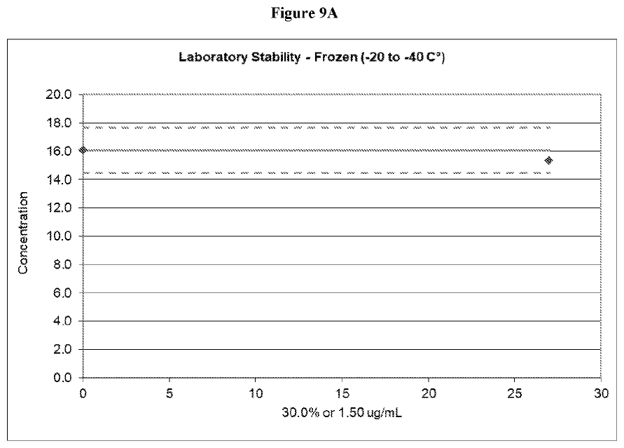

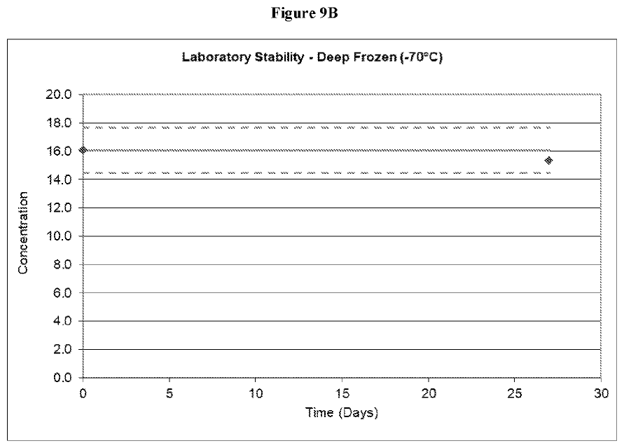

The average concentration was within TEa/3 of the average concentration measured at t=0 for all assessed timepoints (Table 11; FIG. 9). Adiponectin is stable up to 27 days at deep frozen temperature.

TABLE 11
Sample Stability (Deep Frozen)
TEa: 30% or 1.50 μg/mL

| | |
|---|---|
| Deep Frozen (−70° C.) | Days |

| Sample # | 0 | 27 |
|---|---|---|
| 1 | 11.26 | 12.92 |
| 2 | 24.44 | 18.04 |
| 3 | 8.73 | 7.14 |
| 4 | 17.38 | 14.91 |
| 5 | 10.70 | 12.95 |
| 6 | 7.82 | 6.34 |
| 7 | 8.54 | 9.11 |
| 8 | 39.77 | 40.19 |
| 9 | 6.52 | 5.94 |
| 10 | 25.66 | 26.07 |
| Average | 16.08 | 15.36 |

| | |
|---|---|
| Target +TEa/3 | 17.69 |
| Day 0 Target | 16.08 |
| Target −TEa/3 | 14.47 |

### Freeze/Thaw Stability

Samples were frozen and thawed for 0, 1, 2, 3, 4, and 5 times.

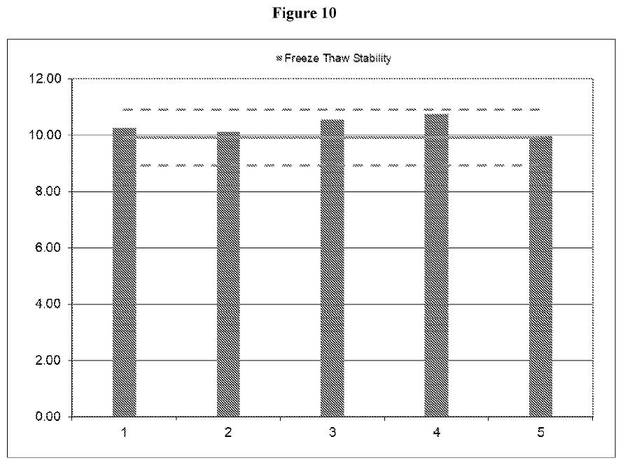

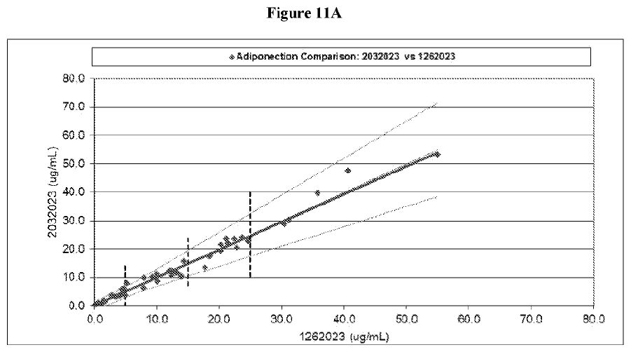

The average concentration was within TEa/3 of the average concentration measured at t=0 for all assessed freeze/thaw cycles (Table 12; FIG. 10). Adiponectin is stable up to 5 freeze-thaw cycles.

TABLE 12
Sample Stability (Freeze/Thaw)
TEa: 30% or 1.50 μg/mL

| | Original | Freeze Thaw | | | | |
|---|---|---|---|---|---|---|
| Sample | 0 | 1 | 2 | 3 | 4 | 5 |
| 1 | 12.96 | 12.84 | 12.23 | 12.69 | 13.04 | 13.66 |
| 2 | 7.12 | 8.76 | 8.1 | 5.02 | 6.77 | 6.92 |
| 3 | 11.76 | 12.52 | 12.68 | 15.93 | 12.98 | 13.79 |
| 4 | 6.35 | 7.08 | 8.92 | 6.98 | 7.41 | 5.02 |
| 5 | 1.54 | 0.8 | 1.48 | 1.07 | 0.83 | 1.7 |
| 6 | 28.5 | 26.71 | 27.84 | 31.56 | 27.21 | 25.9 |
| 7 | 1.11 | 1.03 | 1.15 | 1.42 | 1.32 | 1.43 |
| 8 | 3.35 | 3.4 | 3.19 | 2.37 | 2.76 | 2.29 |
| 9 | 1.13 | 1.11 | 0.64 | 1.54 | 1.15 | 0.95 |
| 10 | 25.36 | 28.42 | 24.98 | 26.89 | 34.03 | 28.05 |
| Average | 9.92 | 10.27 | 10.12 | 10.55 | 10.75 | 9.97 |
| Difference | | 0.35 | 0.20 | 0.63 | 0.83 | 0.05 |
| TEa/3 | | 0.99 | 0.99 | 0.99 | 0.99 | 0.99 |
| Diff <= Tea/3 | | YES | YES | YES | YES | YES |

### Autosampler Stability

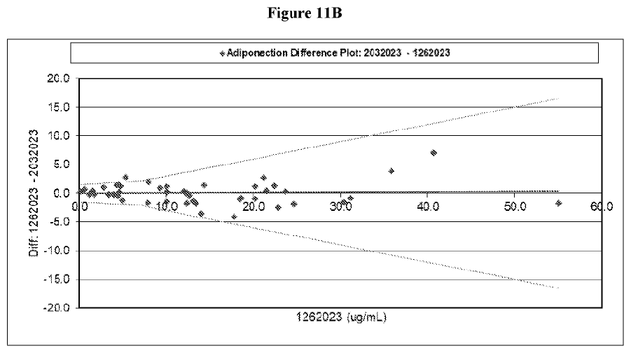

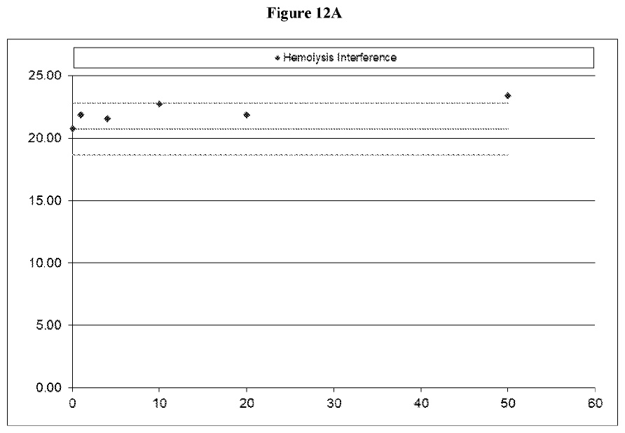

47 samples were processed and assayed on day 0 and 6 to assess stability of prepared samples in the instrument autosampler. The data supporting this study is shown in Table 13, FIG. 11. All reported parameters remained within acceptance criteria for the duration of the study.

TABLE 13
Autosampler Stability
TEa: 30% or 1.50 μg/mL

| | | Day | Day | | | |
|---|---|---|---|---|---|---|
| Sample | Sample | 0 | 6 | Difference, | Difference, | |
| # | Type | (X) | (Y) | Y − X | (Y − X)/X % | AMR* |
| 1 | Serum | 0.20 | 0.55 | 0.35 | 175% | L |
| 2 | Serum | 0.64 | 1.29 | 0.65 | 102% | L |
| 3 | Serum | 1.17 | 0.96 | −0.21 | −18% | L |
| 4 | Serum | 4.02 | 3.84 | −0.18 | −4% | |
| 5 | Serum | 7.91 | 6.25 | −1.66 | −21% | |
| 6 | Serum | 13.97 | 10.38 | −3.59 | −26% | |
| 7 | Serum | 31.16 | 30.33 | −0.83 | −3% | |
| 8 | Serum | 55.03 | 53.23 | −1.80 | −3% | H |
| 9 | Serum | 24.65 | 22.74 | −1.91 | −8% | |
| 10 | Serum | 22.40 | 23.76 | 1.36 | 6% | |
| 11 | Serum | 22.84 | 20.37 | −2.47 | −11% | |
| 12 | Serum | 20.25 | 19.35 | −0.90 | −4% | |
| 13 | Serum | 23.66 | 24.06 | 0.40 | 2% | |
| 14 | Serum | 21.13 | 23.78 | 2.65 | 13% | |
| 15 | Serum | 18.46 | 17.46 | −1.00 | −5% | |
| 16 | Serum | 13.08 | 11.77 | −1.31 | −10% | |
| 17 | Serum | 12.67 | 12.34 | −0.33 | −3% | |
| 18 | Serum | 12.26 | 12.42 | 0.16 | 1% | |
| 19 | Serum | 10.11 | 10.32 | 0.21 | 2% | |
| 20 | Serum | 13.36 | 11.65 | −1.71 | −13% | |
| 21 | Serum | 12.41 | 10.62 | −1.79 | −14% | |
| 22 | Serum | 12.02 | 12.41 | 0.39 | 3% | |
| 23 | Serum | 4.50 | 4.17 | −0.33 | −7% | |
| 24 | Serum | 3.36 | 3.24 | −0.12 | −4% | |
| 25 | Serum | 5.30 | 8.10 | 2.80 | 53% | |
| 26 | Serum | 2.80 | 3.80 | 1.00 | 36% | |
| 27 | Serum | 4.62 | 4.92 | 0.30 | 6% | |
| 28 | Serum | 4.55 | 6.08 | 1.53 | 34% | |
| 29 | Serum | 4.35 | 5.82 | 1.47 | 34% | |
| 30 | Serum | 14.33 | 15.80 | 1.47 | 10% | |
| 31 | Serum | 10.10 | 8.62 | −1.48 | −15% | |
| 32 | Serum | 5.00 | 3.74 | −1.26 | −25% | |
| 33 | Serum | 9.27 | 10.21 | 0.94 | 10% | |
| 34 | Serum | 17.77 | 13.71 | −4.06 | −23% | |
| 35 | Serum | 12.31 | 12.48 | 0.17 | 1% | |
| 36 | Serum | 7.98 | 9.90 | 1.92 | 24% | |
| 37 | Serum | 21.47 | 21.94 | 0.47 | 2% | |
| 38 | Serum | 30.40 | 28.90 | −1.50 | −5% | |
| 39 | Serum | 18.56 | 17.73 | −0.83 | −4% | |
| 40 | Serum | 20.24 | 21.45 | 1.21 | 6% | |
| 41 | Serum | 10.05 | 11.23 | 1.18 | 12% | |
| 42 | Serum | 35.83 | 39.71 | 3.88 | 11% | |
| 43 | Serum | 4.67 | 5.94 | 1.27 | 27% | |
| 44 | Serum | 1.51 | 1.96 | 0.45 | 30% | |
| 45 | Serum | 40.69 | 47.71 | 7.02 | 17% | |
| 46 | Serum | 4.79 | 6.03 | 1.24 | 26% | |
| 47 | Serum | 1.76 | 1.56 | −0.20 | −11% | |

*L: a value below the lowest calibration standard (below the analytical measurement range) was recovered; H: a value above the highest calibration standard (above the analytical measurement range) was recovered.

Processed samples stored in the autosampler for this assay are stable for 6 days.

### Example 9

Analytical Specifity-Interfering Substances. Acceptance criteria for interfering substance studies is that the average of specimen results do not deviate from the unaltered measurement by more than TEa/3.

### Hemolysis

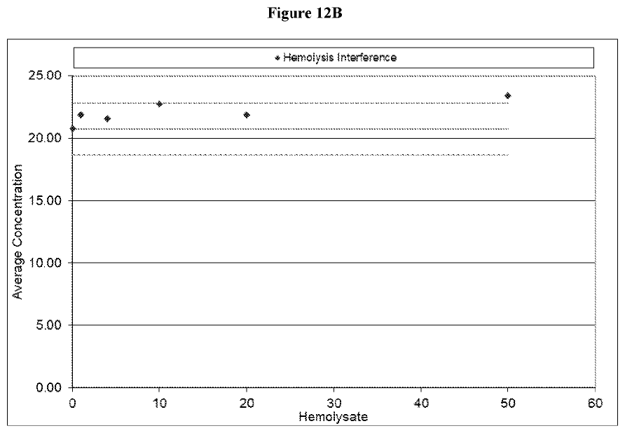

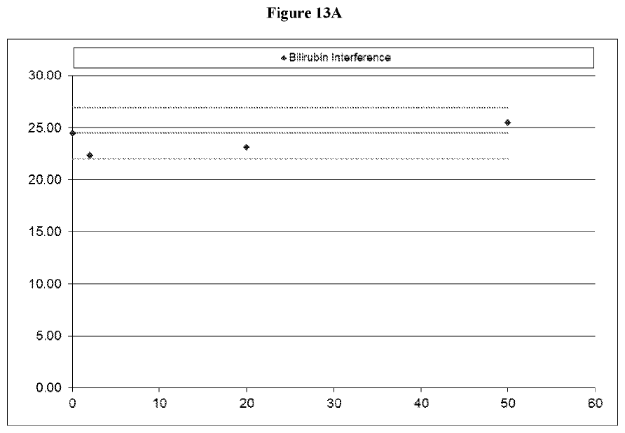

Single donor serum pools were spiked with red blood cell hemolysate to concentrations of 0, 10, 40, 100, 200, and 500 mg/dL and run-in quadruplicate. Samples were assayed at each level and the results are presented in Tables 14 and FIG. 12. All reported parameters remained within acceptance criteria except for the highest tested concentration of interferant. Gross hemolysis samples are not acceptable for testing.

TABLE 14
Hemolysis
TEa: 30% or 1.50 μg/mL

| | |
|---|---|
| Hemolysis | mg/dL |

| Sample | 0 | 10 | 40 | 100 | 200 | 500 |
|---|---|---|---|---|---|---|
| 1 | 29.24 | 32.25 | 32.34 | 34.38 | 33.30 | 34.91 |
| 2 | 2.77 | 2.67 | 3.14 | 3.39 | 3.69 | 3.87 |
| 3 | 32.25 | 31.29 | 32.37 | 32.66 | 30.55 | 35.79 |
| 1 | 24.99 | 32.12 | 29.91 | 35.73 | 31.21 | 32.70 |
| 2 | 2.99 | 3.26 | 3.31 | 3.08 | 3.28 | 4.34 |
| 3 | 30.71 | 29.85 | 29.86 | 29.76 | 31.12 | 29.93 |
| 1 | 30.97 | 34.05 | 29.17 | 35.27 | 29.65 | 31.83 |
| 2 | 3.79 | 3.09 | 3.99 | 2.84 | 4.22 | 4.15 |
| 3 | 29.45 | 29.80 | 30.11 | 29.47 | 28.53 | 33.25 |
| 1 | 27.60 | 30.66 | 32.43 | 31.88 | 30.53 | 33.68 |
| 2 | 3.03 | 3.03 | 2.85 | 3.65 | 3.78 | 5.30 |
| 3 | 31.01 | 30.58 | 29.03 | 30.97 | 32.80 | 30.64 |
| Average | 20.73 | 21.89 | 21.54 | 22.76 | 21.89 | 23.37 |
| X + Tea/3 | 22.8 | 22.8 | 22.8 | 22.8 | 22.8 | 22.8 |
| X | 20.7 | 20.7 | 20.7 | 20.7 | 20.7 | 20.7 |
| X − Tea/3 | 18.7 | 18.7 | 18.7 | 18.7 | 18.7 | 18.7 |

### Icterus (Bilirubin)

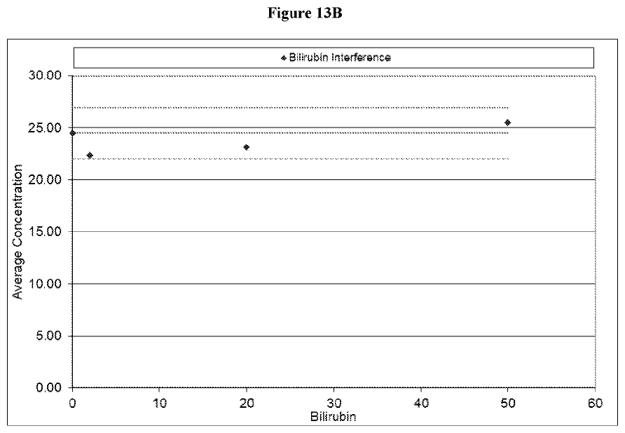

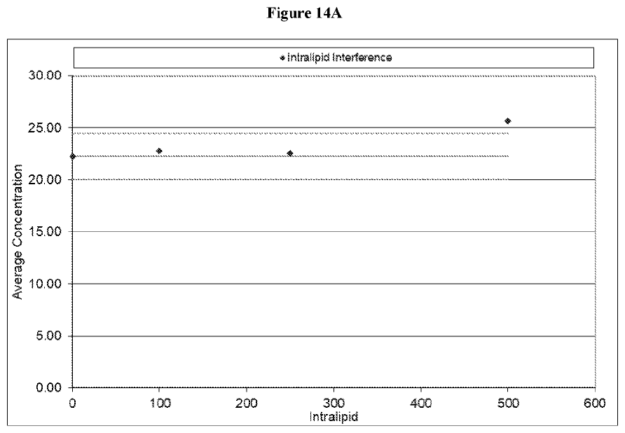

Single donor serum pools were spiked with bilirubin to concentrations of 0, 0.2, 2 and 5 mg/dL and run-in quadruplicate. Samples were assayed at each level and the results are presented in Tables 15 and FIG. 13. All reported parameters remained within acceptance criteria up to the highest tested concentration of interferant.

TABLE 15
Bilirubin
TEa: 30% or 1.50 μg/mL

| | |
|---|---|
| Bilirubin | mg/dL |

| Sample | 0 | 0.2 | 2 | 5 |
|---|---|---|---|---|
| 1 | 38.30 | 36.11 | 34.25 | 39.90 |
| 2 | 3.89 | 3.45 | 4.05 | 3.92 |
| 3 | 34.24 | 32.69 | 35.05 | 35.66 |
| 1 | 39.85 | 35.00 | 36.28 | 37.86 |
| 2 | 3.80 | 2.87 | 4.32 | 8.75 |
| 3 | 34.80 | 27.73 | 29.32 | 35.24 |
| 1 | 31.39 | 35.22 | 38.95 | 37.87 |
| 2 | 3.44 | 2.29 | 3.32 | 5.54 |
| 3 | 33.25 | 24.96 | 31.46 | 32.18 |
| 1 | 36.79 | 36.18 | 56.66 | 34.49 |
| 2 | 3.92 | 3.63 | 3.60 | 2.81 |
| 3 | 30.17 | 27.93 | 33.93 | 31.72 |
| Average | 24.49 | 22.34 | 23.14 | 25.50 |
| X +Tea/3 | 26.9 | 26.9 | 26.9 | 26.9 |
| X | 24.5 | 24.5 | 24.5 | 24.5 |
| X −Tea/3 | 22.0 | 22.0 | 22.0 | 22.0 |

### Lipemia (Intralipid)

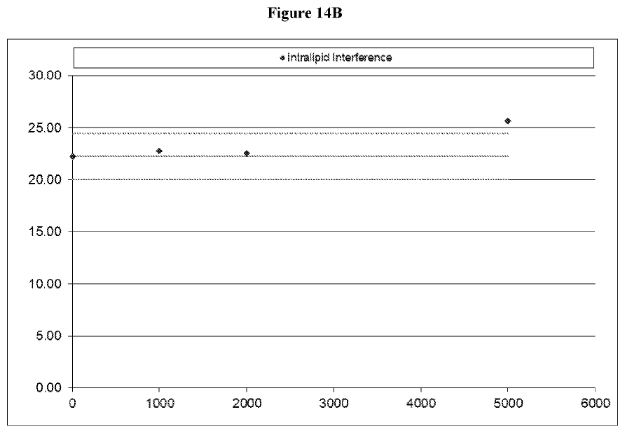

Single donor serum pools were spiked with intralipid to concentrations of 0, 100, 250 and 500 mg/dL and run-in quadruplicate. Samples were assayed at each level and the results are presented in Table 16 and FIG. 14. All reported parameters remained within acceptance criteria except for the highest tested concentration of interferant. Gross lipemic samples are not acceptable for testing.

TABLE 16
Intralipid
TEa: 30% or 1.50 μg/mL

| | |
|---|---|
| Intralipid | mg/dL |

| Sample | 0 | 100 | 250 | 500 |
|---|---|---|---|---|
| 1 | 37.59 | 32.92 | 37.57 | 39.85 |
| 2 | 2.71 | 2.65 | 3.30 | 3.60 |
| 3 | 31.71 | 34.62 | 38.45 | 38.44 |
| 1 | 33.66 | 30.24 | 31.66 | 37.50 |
| 2 | 3.48 | 4.22 | 2.59 | 3.18 |
| 3 | 29.21 | 32.21 | 28.65 | 35.37 |
| 1 | 30.40 | 31.08 | 31.44 | 34.62 |
| 2 | 3.21 | 3.54 | 3.84 | 2.32 |
| 3 | 28.70 | 33.07 | 28.08 | 38.86 |
| 1 | 32.57 | 31.55 | 32.62 | 33.84 |
| 2 | 3.55 | 2.87 | 3.00 | 2.91 |
| 3 | 30.16 | 34.68 | 29.03 | 37.04 |
| Average | 22.25 | 22.80 | 22.52 | 25.63 |
| X +Tea/3 | 24.5 | 24.5 | 24.5 | 24.5 |
| X | 22.2 | 22.2 | 22.2 | 22.2 |
| X −Tea/3 | 20.0 | 20.0 | 20.0 | 20.0 |

### Example 10

Reportable Range. The reportable range of this assay is 1.50 to 50.00 μg/mL.

### Example 11

Reference Interval. The reference range for this assay (noted in the table below) will be adopted by comparison from the currently validated ELISA assay, as split sample comparison between the candidate LC-MS method and the validated ELISA method compared across the reference interval within TEa/4.

| | | |
|---|---|---|
| BMI | Male | Female |

| | | | |
|---|---|---|---|
| <25 | kg/m² | 4-26 μg/mL | 5-37 μg/mL |
| 25-30 | kg/m² | 4-20 μg/mL | 5-28 μg/mL |
| >30 | kg/m² | 2-20 μg/mL | 4-22 μg/mL |

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

While certain embodiments have been illustrated and described, it should be understood that changes and modifications can be made therein in accordance with ordinary skill in the art without departing from the technology in its broader aspects as defined in the following claims.

1. A method for determining an amount of adiponectin in a sample by mass spectrometry, the method comprising:
(a) subjecting adiponectin from a sample to an ionization source under conditions suitable to generate one or more adiponectin ions detectable by mass spectrometry;
(b) determining the amount of one or more adiponectin ions by mass spectrometry; and
(c) determining the amount of adiponectin in the sample from the amount of one or more adiponectin ions determined in step (b).

2. The method of claim 1, wherein the sample comprises a plasma or serum sample.

3. The method of claim 1, wherein the ionization source is an electrospray (ESI) ionization source.

4. The method of claim 1, wherein the ionization is in positive mode.

5. The method of claim 1, wherein the one or more adiponectin ions comprise an ion that has a mass to charge ratio (m/z) of 314.18±0.5.

6. The method of claim 1, wherein the one or more adiponectin ions comprise an ion that has a mass to charge ratio (m/z) of 627.36±0.5

7. The method of claim 1, wherein the one or more adiponectin ions comprise an ion that has a mass to charge ratio (m/z) of 530.30±0.5.

8. The method of claim 1, wherein an internal standard for adiponectin is added to the sample.

9. The method of claim 8, wherein the internal standard is recombinant human adiponectin or adiponectin peptides.

10. The method of claim 8, wherein the method comprises generating one or more ions of the internal standard that are detectable by mass spectrometry.

11. The method of claim 10, wherein the one or more ions of the internal standard comprise an ion that has a mass to charge ratio (m/z) of 319.19±0.5.

12. The method of claim 10, wherein the one or more ions of the internal standard comprise an ion that has a mass to charge ratio (m/z) of 637.37±0.5.

13. The method of claim 10, wherein the one or more ions of the internal standard comprise an ion that has a mass to charge ratio (m/z) of 540.31±0.5.

14. The method of claim 1, further comprising digestion with trypsin.

15. The method of claim 1, further comprising precipitation with formic acid.

16. The method of claim 1, further comprising purifying the sample prior to mass spectrometry.

17. The method of claim 16, wherein the purifying comprises subjecting the sample to liquid chromatography.

18. The method of claim 17, wherein the liquid chromatography comprises high performance liquid chromatography (HPLC).

19. The method of claim 1, wherein the mass spectrometry is tandem mass spectrometry.

20. The method of claim 1, wherein a low level of adiponectin corresponds to increased risk of metabolic syndrome or type 2 diabetes.

21. (canceled)
